(19) United States
(12) Patent Application Publication
(10) Pub. No.: US 2022/0380825 A1
(43) Pub. Date: Dec. 1, 2022

(54) METHODS FOR ENHANCED BIOSYNTHESIS AND SCREENING OF ANTIBIOTICS

(71) Applicant: Inscripta, Inc., Boulder, CO (US)

(72) Inventors: Tyson Shepherd, Boulder, CO (US); Steve Federowicz, Boulder, CO (US); Dan Held, Boulder, CO (US)

(21) Appl. No.: 17/683,343

(22) Filed: Feb. 28, 2022

Related U.S. Application Data
(60) Provisional application No. 63/155,706, filed on Mar. 2, 2021.

(51) Int. Cl. C12P 21/00; C12N 15/10; C12N 1/20; C12N 15/70

(52) U.S. Cl. CPC C12P 21/00; C12N 15/1058; C12N 1/205; C12N 15/70; C12R 2001/19

(57) ABSTRACT

The present disclosure is directed to devices, instruments, and methods, including automated methods, for enhanced production and efficient screening of biosynthesized antibiotics. More particularly, the present disclosure provides for accelerated biosynthesis and screening on a single-cell scale.

### CROSS-REFERENCE TO RELATED APPLICATIONS

The present application claims priority to U.S. Ser. No. 63/155,706, filed Mar. 2, 2021, which is herein incorporated by reference in its entirety.

### FIELD OF THE INVENTION

The present disclosure relates to methods and compositions for rapidly synthesizing and screening antibiotics that overcome widespread antibiotic resistance, as well as devices and systems for performing these methods and using these compositions.

### BACKGROUND OF THE INVENTION

In the following discussion, certain articles and methods will be described for background and introductory purposes. Nothing contained herein is to be construed as an “admission” of prior art. Applicant expressly reserves the right to demonstrate, where appropriate, that the articles and methods referenced herein do not constitute prior art under the applicable statutory provisions.

Antibiotics have revolutionized the treatment of infectious diseases since the original discovery of penicillin in 1928. Prior to the 20^{th} century, infectious diseases accounted for high morbidity and mortality worldwide. However, with the advent of antibiotics, the leading causes of death began to shift from communicable diseases to non-communicable diseases such as cardiovascular disease, stroke, and cancer.

A significant threat to the utility and efficacy of antibiotics is antibiotic resistance, which is the ability of bacteria to resist the effect of antibiotics to which they were initially sensitive. Most pathogenic bacteria have the capability of developing resistance to at least some antibacterial agent, and many strains have developed resistance to multiple antibacterial agents. Generally, the main mechanisms of antibiotic resistance include limiting uptake of an antibiotic compound, modification or overproduction of an antibiotic target (e.g., a targeted protein), inactivation of an antibiotic compound (e.g., by breaking of chemical bonds), bypassing an antibiotic target or pathway, and active efflux (e.g. pumping out to reduce internal concentration) of an antibiotic compound. These mechanisms may be native to the bacteria or acquired from other microorganisms, and may further result from selection pressure from antibiotic use (including overuse) that provides a competitive advantage for mutated strains.

Because resistant bacteria are difficult to treat, thus requiring higher doses or alternative antibiotics, there is a pressing need for rapid development of new antibiotics that overcome widespread antibiotic resistance. Current state-of-the-art antibiotic screening methods include multistep processes that can be broadly characterized into two segments: synthesis of new compounds and microtiter plate-based screening for antibiotic efficacy.

In the first synthesis step, antibiotic compounds may be chemically synthesized or biologically produced, and thereafter purified. Prototypical biological production pathways (e.g., enzymatic pathways) leading to efficacious and widely used compounds have been found in Streptomyces and Bacillus bacterial species, as well as other microorganisms such as fungi, yeast, and plants. The microorganism-produced antibiotic compounds are generated as a result of an ongoing arms race between microbes and thus, the antibiotic compounds will typically target essential proteins of other organisms while leaving the source organism unaffected. For example, a Streptomyces species may produce the antibiotic streptomycin for release to the local environment, which may be taken up by nearby E. coli, binding to the 16S rRNA of the 30S ribosome subunit thereof and blocking initiation of E. coli proteins. Thus, the Streptomyces species may win the local race for resources (e.g., nutrients) by producing and releasing streptomycin.

After synthesis and, in certain instances, separation and purification, the antibiotic compounds may be screened to identify compounds that can be reliably produced to high quality and efficacy. Antibiotic efficacy is formally defined as the minimum inhibitory concentration (“MIC”), or the compound concentration in a cultivation media at which no observable microbial growth occurs. To screen for antibiotic compounds, a typical experiment entails growing a target bacterial species in individual wells of a 96-well microtiter. Cell growth in each well is simultaneously monitored by tracking optical densities of the samples using a plate reader (e.g., at a wavelength of 595 nm or 600 nm (OD600)). Antibiotic candidates are then added to designated test wells, and growth rates of the initial inoculums of bacteria are monitored by tracking optical density profiles. Thus, current antibiotic screening methods are onerous and inefficient.

Accordingly, there is a need in the art for improved methods of developing and screening antibiotics that overcome widespread antibiotic resistance. The present disclosure addresses this need.

### SUMMARY OF ILLUSTRATIVE EMBODIMENTS

This Summary is provided to introduce a selection of concepts in a simplified form that are further described below in the Detailed Description. This Summary is not intended to identify key or essential features of the claimed subject matter, nor is it intended to be used to limit the scope of the claimed subject matter. Other features, details, utilities, and advantages of the claimed subject matter will be apparent from the following written Detailed Description including those aspects illustrated in the accompanying drawings and defined in the appended claims.

The present disclosure relates to methods, compositions, and automated multi-module cell processing instruments for the editing of cells for bioproduction of antibiotics that overcome antibiotic resistance. The disclosure includes methods of using nucleic acid-guided editing in cell populations, e.g., bacterial cells, for creation of cell populations that enable enhanced bioproduction and screening of antibiotics.

In some aspects, the disclosure provides cells produced by the disclosed methods, wherein the cells are designed such that each cell genome encodes both an antibiotic production pathway and an antibiotic resistance pathway, thereby the antibiotic screening process to a single-cell scale.

In specific aspects, each cell is engineered to simultaneously encode a single copy of at least one protein that facilitates production of an antibiotic and a single copy of at least one protein that facilitates resistance to the produced antibiotic. The proteins may be encoded as single copies inserted into the genome, or as replicated on a single-copy plasmid such as a bacterial artificial chromosome.

In some aspects, the cells edited to include both antibiotic production genes and antibiotic resistance genes are further edited to produce genetic variants of the antibiotic production genes. Accordingly, in some aspects, the genetic variants of the antibiotic production genes generate potent antibiotic compounds that overcome resistance provided by the co-encoded resistance genes.

In some aspects, cells encoding genetic variants of the antibiotic production genes may be screened and selected by negative selection.

Following production of a potent antibiotic compound, the antibiotic compound can optionally be isolated from the modified cells.

In some aspects, upon production of potent antibiotic compounds overcoming the co-encoded resistance genes, the cells are further edited to produce genetic variants of the resistance genes. Accordingly, in some aspects, the genetic variants of the resistance genes facilitate cell survival in the presence of the potent antibiotic compounds.

In some aspects, cells encoding genetic variants of the resistance genes may be screened and selected by positive selection.

In some aspects, the cells are exposed to multiple rounds of editing of antibiotic production genes, negative selection, editing of resistance genes, and positive selection to produce novel antibiotic compounds and resistance pathways. Accordingly, alternating editing of antibiotic production genes and editing of resistance genes may simulate natural evolution of the cells at a more rapid, single-cell scale.

in some aspects, the cells edited using the methods of the present disclosure are bacterial cells which serve as rapid screening “mills” to produce and screen for novel antibiotics that escape known and novel resistance pathways.

The cells that can be edited using the methods of the disclosure generally include microbial cells, including both prokaryotic cells and unicellular and multicellular filamentous fungi. In some examples, prokaryotic cells for use with the present illustrative embodiments can be gram-positive bacterial cells, e.g., Bacillus or Streptomyces cells, or gram-negative bacterial cells, e.g., Escherichia cells. In some examples, fungal cells for use with the present illustrative embodiments can be yeast cells, e.g., Candida cells.

In some aspects, the cells are engineered to contain a heterologous pathway for the bioproduction of antibiotic compounds and/or resistance to the antibiotic compounds. In specific aspects, the heterologous pathway is introduced into cells of a model organism including but not limited to E. coli, S. cervisiae, Streptomyces, Streptococcus, Pseudomonas, Vibrio natriegens, Bradyrhizobium, Lactobacillus, Bifidobacterium, Bacillus, Pichia pastoris, Candida, Aspergillus, Cryptococcus, Histoplasma, Pneumocystis, Fusarium, Penicillium, and Lomentospora. In specific aspects, heterologous genes encoding for compounds with known antibiotic activity and/or resistance to antibiotic compounds are engineered into cells of common research microbial species such as E. coli.

In some aspects, the methods described herein are utilized to edit cells of non-pathogenic and/or probiotic microorganisms, such as E. coli strain Nissle 1917 (ECN) and Lactobacillus acidophilus, which may be subsequently deposited in a patient gut.

In some aspects, the microbes edited using the methods of the present disclosure include bacterial cells.

In some aspects, the genes of organisms with an ability to produce antibiotics and/or organisms that are resistant to antibiotic compounds (or mutations found in homologous genes from these organisms) can be introduced as heterologous sequences into cells of another genera, species, or strain. Accordingly, in some aspects, genes from one organism are heterologously introduced into cells of another organism for genetic manipulation and construction of cells with both antibiotic production and antibiotic resistance capabilities. The genes could either be integrated into the new host cell genome or carried on a plasmid, such as a bacterial artificial chromosome (BAC).

In some aspects, cells from organisms with known antibiotic activity or resistance activity can be used as “starting” organisms that are further edited to include both antibiotic production and antibiotic resistance pathways.

In certain embodiments, automated methods are used for nuclease-directed genome editing of one or more target genomic regions in multiple cells for bioproduction of novel antibiotics that escape known and novel resistance pathways, the methods being performed in automated multi-module cell editing instruments. These automated methods carried out using the automated multi-module cell editing instruments described herein can be used with a variety of nuclease-directed genome editing techniques, and can be used with or without additional selectable markers.

In further aspects, the methods described herein are applied to other engineered pathways within microbial cells, such as antifungal, anti-phage, and antiviral pathways.

In some aspects, the cells are edited by performing one to many rounds (e.g., iterations) of genomic editing utilizing large libraries of desired edits in each round.

In some aspects, cells are edited utilizing a nucleic acid-guided nuclease editing system. The nucleic acid-guided nuclease editing system may comprise a nuclease or CF enzyme, and an editing cassette to effect editing in live cells.

In some aspects, the editing cassette comprises a gRNA covalently linked to a repair template for effecting an edit. Generally, the gRNA comprises a region of complementarity to a target sequence in which an edit is to be incorporated.

In some aspects, the gRNA is a CREATE fusion gRNA (“CFgRNA,” defined infra), and the editing cassette is a CREATE fusion editing cassette (“CF editing cassette,” defined infra) comprising from 5′ to 3′: 1) the CFgRNA having a region of complementarity to a target sequence in which an edit is to be incorporated, the CFgRNA comprising: a guide or spacer sequence, and a scaffold region recognized by a corresponding nuclease or nickase; and 2) a repair template covalently linked to the CFgRNA and comprising an edit.

In some aspects, the editing cassette further comprises an RNA G-quadruplex region at a 3′ end of the repair template to stabilize the cassette and improve target nicking or cleavage efficiency without inducing off-target activity.

In some aspects, the editing cassette further comprises an amplification priming site or subpool primer binding sequence at a 3′ end thereof. In specific aspects, the editing cassette further comprises a melting temperature booster sequence at a 5′ end thereof, which is a short protective DNA buffer sequence. In addition, in specific aspects, the editing cassette comprises regions of homology to a vector for gap-repair insertion of the cassette into the vector, such as an editing vector or engine vector.

In some aspects, the editing cassette further comprises a barcode sequence.

In some aspects, the nuclease includes a MAD-series nuclease, nickase, or a variant (e.g., orthologue) thereof. In some aspects, the nuclease includes a MAD1, MAD2, MAD3, MAD4, MAD5, MAD6, MAD7, MAD8, MAD9, MAD10, MAD11, MAD12, MAD13, MAD14, MAD15, MAD16, MAD17, MAD18, MAD19, MAD20, MAD2001, MAD2007, MAD2008, MAD2009, MAD2011, MAD2017, MAD2019, MAD297, MAD298, MAD299, or other MAD-series nuclease, nickase, variants thereof, and/or combinations thereof.

In some aspects, the nuclease includes a Cas9 nuclease (also known as Csn1 and Csx12), nickase, or a variant thereof.

In some aspects, the nuclease includes C2c1, C2c2, C2c3, Cas1, Cas1B, Cas2, Cas3, Cas4, Cas5, Cas6, Cas7, Cas8, Cas10, Cpf1, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx100, Csx16, CsaX, Csx3, Csx1, Csx15, Csf1, Csf2, Csf3, Csf4, or similar nuclease, nickase, variants thereof, and/or combinations thereof.

In some aspects, such as embodiments wherein a CF editing cassette is utilized, the nuclease is a fusion protein—i.e., a nucleic acid-guided nickase/reverse transcriptase fusion enzyme (a “nickase-RT fusion”)—that retains certain characteristics of nucleic acid-directed nucleases (e.g., the binding specificity and ability to cleave one or more DNA strands in a targeted manner) combined with another enzymatic activity, namely, reverse transcriptase activity. The reverse transcriptase portion of the nickase-RT fusion may use a repair template of an editing cassette to synthesize and edit at a “flap” created by the nickase portion on one or both DNA strands of a target editing locus, thereby circumventing endogenous mismatch repair systems to incorporate an edit.

The present disclosure thus provides, in selected embodiments, modules, instruments, and systems for automated multi-module cell editing for bioproduction of novel antibiotics that escape known and novel resistance pathways. Automated systems for cell editing that may be used can be found, e.g., in U.S. Pat. Nos. 10,253,316; 10,329,559; 10,323,242; 10,421,959; 10,465,185; 10,519,437; 10,584,333; 10,584,334; 10,647,982; 10,689,645; 10,738,301; and 10,738,663.

The aspects and other features and advantages of the invention are described below in more detail.

### DETAILED DESCRIPTION

All of the functionalities described in connection with one embodiment of the methods, devices or instruments described herein are intended to be applicable to the additional embodiments of the methods, devices and instruments described herein except where expressly stated or where the feature or function is incompatible with the additional embodiments. For example, where a given feature or function is expressly described in connection with one embodiment but not expressly mentioned in connection with an alternative embodiment, it should be understood that the feature or function may be deployed, utilized, or implemented in connection with the alternative embodiment unless the feature or function is incompatible with the alternative embodiment.

The practice of the techniques described herein may employ, unless otherwise indicated, conventional techniques and descriptions of molecular biology (including recombinant techniques), cell biology, biochemistry, and genetic engineering technology, which are within the skill of those who practice in the art. Such conventional techniques and descriptions can be found in standard laboratory manuals such as Green and Sambrook, Molecular Cloning: A Laboratory Manual. 4th, ed., Cold Spring Harbor Laboratory Press, Cold Spring Harbor, N.Y., (2014); Current Protocols in Molecular Biology, Ausubel, et al. eds., (2017); Neumann, et al., Electroporation and Electrofusion in Cell Biology, Plenum Press, New York, 1989; and Chang, et al., Guide to Electroporation and Electrofusion, Academic Press, California (1992), all of which are herein incorporated in their entirety by reference for all purposes. Nucleic acid-guided nuclease techniques can be found in, e.g., Genome Editing and Engineering from TALENs and CRISPRs to Molecular Surgery, Appasani and Church (2018); and CRISPR: Methods and Protocols, Lindgren and Charpentier (2015); both of which are herein incorporated in their entirety by reference for all purposes.

Note that as used herein and in the appended claims, the singular forms “a,” “an,” and “the” include plural referents unless the context clearly dictates otherwise. Thus, for example, reference to “a cell” refers to one or more cells, and reference to “the system” includes reference to equivalent steps, methods and devices known to those skilled in the art, and so forth. Additionally, it is to be understood that terms such as “left,” “right,” “top,” “bottom,” “front,” “rear,” “side,” “height,” “length,” “width,” “upper,” “lower,” “interior,” “exterior,” “inner,” “outer” that may be used herein merely describe points of reference and do not necessarily limit embodiments of the present disclosure to any particular orientation or configuration. Furthermore, terms such as “first,” “second,” “third,” etc., merely identify one of a number of portions, components, steps, operations, functions, and/or points of reference as disclosed herein, and likewise do not necessarily limit embodiments of the present disclosure to any particular configuration or orientation.

Unless defined otherwise, all technical and scientific terms used herein have the same meaning as commonly understood by one of ordinary skill in the art to which this invention belongs. All publications mentioned herein are incorporated by reference for all purposes, including but not limited to describing and disclosing devices, formulations and methodologies that may be used in connection with the presently described invention.

Where a range of values is provided, it is understood that each intervening value, between the upper and lower limit of that range and any other stated or intervening value in that stated range is encompassed within the invention. The upper and lower limits of these smaller ranges may independently be included in smaller ranges, and are also encompassed within the invention, subject to any specifically excluded limit in the stated range. Where the stated range includes one or both of the limits, ranges excluding either or both of those included limits are also included in the invention.

In the following description, numerous specific details are set forth to provide a more thorough understanding of the present invention. However, it will be apparent to one of skill in the art that the present invention may be practiced without one or more of these specific details. In other instances, features and procedures well known to those skilled in the art have not been described in order to avoid obscuring the invention. The terms used herein are intended to have the plain and ordinary meaning as understood by those of ordinary skill in the art.

The term “amino acid” refers to naturally occurring and synthetic amino acids, as well as amino acid analogs that function similarly to naturally occurring amino acids.

The terms “cassette,” “expression cassette,” “editing cassette,” “CREATE cassette,” “CREATE editing cassette,” “CREATE fusion editing cassette,” or “CF editing cassette” in the context of the current methods and compositions refer to a nucleic acid molecule comprising a coding sequence for transcription of a guide nucleic acid (gRNA) to facilitate editing of one or both DNA strands in a nucleic acid-guided nuclease system. In certain embodiments, “CF editing cassette” refers to a nucleic acid molecule comprising a coding sequence for transcription of a guide nucleic acid or gRNA covalently linked to a coding sequence for transcription of a repair template.

The term “complementary” as used herein refers to Watson-Crick base pairing between nucleotides and specifically refers to nucleotides hydrogen-bonded to one another with thymine or uracil residues linked to adenine residues by two hydrogen bonds and cytosine and guanine residues linked by three hydrogen bonds. In general, a nucleic acid includes a nucleotide sequence described as having a “percent complementarity” or “percent homology” to a specified second nucleotide sequence. For example, a nucleotide sequence may have 80%, 90%, or 100% complementarity to a specified second nucleotide sequence, indicating that 8 of 10, 9 of 10 or 10 of 10 nucleotides of a sequence are complementary to the specified second nucleotide sequence. For instance, the nucleotide sequence 3′-TCGA-5′ is 100% complementary to the nucleotide sequence 5′-AGCT-3′; and the nucleotide sequence 3′-TCGA-5′ is 100% complementary to a region of the nucleotide sequence 5′-TTAGCTGG-3′.

The term DNA “control sequences” refers collectively to promoter sequences, polyadenylation signals, transcription termination sequences, upstream regulatory domains, origins of replication, internal ribosome entry sites, nuclear localization sequences, enhancers, and the like, which collectively provide for the replication, transcription and translation of a coding sequence in a recipient cell. Not all of these types of control sequences need to be present so long as a selected coding sequence is capable of being replicated, transcribed and—for some components—translated in an appropriate host cell.

The terms “CREATE fusion gRNA” or “CFgRNA” refer to a gRNA engineered to function with a nucleic acid-guided nickase/reverse transcriptase fusion enzyme (a “nickase-RT fusion”) where the CFgRNA is designed to bind to and facilitate editing of one or both DNA strands in a target locus of a cell genome. In certain embodiments, “CREATE fusion gRNA” or “CFgRNA” refer to one of two gRNAs engineered to function with a nucleic acid-guided nickase/reverse transcriptase fusion enzyme (a “nickase-RT fusion”) where the two CFgRNAs are designed to bind to and edit/barcode opposite DNA strands in a target locus. The two CFgRNAs specific to a target locus have regions of complementarity to one another at least at the site of the edit and preferably at regions 5′ and 3′ to the site of the edit. The term “complementary CFgRNAs” refers to two CFgRNAs engineered to bind to opposite DNA strands in a target locus which often create the complementary edit at a site in the target locus.

The terms “CREATE fusion editing system” or “CF editing system” refer to the combination of a nucleic acid-guided nickase enzyme/reverse transcriptase fusion protein (“nickase-RT fusion”) and a CREATE fusion editing cassette (“CF editing cassette”) to effect editing in live cells. In certain embodiments, a CF editing system further includes a CREATE fusion barcoding cassette (“CF barcoding cassette”).

As used herein the term “donor DNA” or “donor nucleic acid” refers to nucleic acid that is designed to introduce a DNA sequence modification (insertion, deletion, substitution) into a locus (e.g., a target genomic DNA sequence or cellular target sequence) by homologous recombination using nucleic acid-guided nucleases. For homology-directed repair, the donor DNA must have sufficient homology to the regions flanking the “cut site” or site to be edited in the genomic target sequence. The length of the homology arm(s) will depend on, e.g., the type and size of the modification being made. In many instances and preferably, the donor DNA will have two regions of sequence homology (e.g., two homology arms) to the genomic target locus. Preferably, an “insert” region or “DNA sequence modification” region—the nucleic acid modification that one desires to be introduced into a genome target locus in a cell-will be located between two regions of homology. The DNA sequence modification may change one or more bases of the target genomic DNA sequence at one specific site or multiple specific sites. A change may include changing 1, 2, 3, 4, 5, 10, 15, 20, 25, 30, 35, 40, 50, 75, 100, 150, 200, 300, 400, or 500 or more base pairs of the genomic target sequence. A deletion or insertion may be a deletion or insertion of 1, 2, 3, 4, 5, 10, 15, 20, 25, 30, 40, 50, 75, 100, 150, 200, 300, 400, or 500 or more base pairs of the genomic target sequence.

As used herein, “enrichment” refers to enriching for edited cells by singulation, inducing editing, and growth of singulated cells into terminal-sized colonies (e.g., saturation or normalization of colony growth).

The term “gene” refers to a segment of DNA involved in producing a polypeptide chain. It may include regions preceding and following a coding region (leader and trailer, respectively), as well as intervening sequences (introns) between individual coding segments (exons).

The terms “guide nucleic acid” or “guide RNA” or “gRNA” refer to a polynucleotide comprising 1) a guide sequence capable of hybridizing to a genomic target locus, and 2) a scaffold sequence capable of interacting or complexing with a nucleic acid-guided nuclease. In certain embodiments herein, a “guide nucleic acid” or “guide RNA” or “gRNA” is a CFgRNA.

The term “heterologous” refers to the relationship between two or more nucleic acids or protein sequences from different sources, or the relationship between a protein (or nucleic acid) and a host cell from different sources. For example, if the combination of a nucleic acid and a host cell is usually not naturally occurring, the nucleic acid is heterologous to the host cell. A particular sequence is “heterologous” to the cell or organism into which it is inserted.

“Homology” or “identity” or “similarity” refers to sequence similarity between two peptides or, more often in the context of the present disclosure, between two nucleic acid molecules. The term “homologous region” or “homology arm” refers to a region on the donor DNA with a certain degree of homology with the target genomic DNA sequence. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences.

The terms “intermediate compound” and “intermediates” refer to a product of a synthesis pathway that is not the terminal product, but which is useful for the production of the final intended product, e.g., an antibiotic. The term “biosynthesized” when used in reference to an antibiotic refers to a chemical compound or substance produced by a living organism, e.g., bacteria. In the broadest sense, biosynthesized antibiotics include any substance produced by life, that is active against bacteria.

The term “nucleic acid” or “polynucleotide” refers to deoxyribonucleic acids (DNA) or ribonucleic acids (RNA) and polymers thereof in either single- or double-stranded form. Unless otherwise indicated, the terms encompass nucleic acids containing known analogues or natural nucleotides that have similar binding properties as the reference nucleic acid and are metabolized in a manner similar to naturally occurring nucleotides. Unless otherwise indicated, in addition to the sequence specifically stated, a particular nucleic acid sequence also implicitly encompasses conservatively modified variants thereof (e.g., degenerate codon substitutions), alleles, orthologues, SNPs, and complementary sequences. The term nucleic acid is used interchangeably with DNA, RNA, cDNA, gene, and mRNA encoded by a gene.

As used herein, “nucleic acid-guided nickase/reverse transcriptase fusion” or “nickase-RT fusion” refers to a nucleic acid-guided nickase—or nucleic acid-guided nuclease or CRISPR nuclease that has been engineered to act as a nickase rather than a nuclease that initiates double-stranded DNA breaks—where the nucleic acid-guided nickase is fused to a reverse transcriptase, which is an enzyme used to generate cDNA from an RNA template. In certain embodiments, “nucleic acid-guided nickase/reverse transcriptase fusion” or “nickase-RT fusion” refers to two or more nucleic acid-guided nickases—or nucleic acid-guided nucleases or CRISPR nucleases that have been engineered to act as nickases rather than nucleases that initiate double-stranded DNA breaks—where the nucleic acid-guided nickases are fused to a reverse transcriptase. For information regarding nickase-RT fusions see, e.g., U.S. Pat. No. 10,689,669 and U.S. Ser. No. 16/740,421.

“Operably linked” refers to an arrangement of elements where the components so described are configured so as to perform their usual function. Thus, control sequences operably linked to a coding sequence are capable of effecting the transcription, and in some cases, the translation, of a coding sequence. The control sequences need not be contiguous with the coding sequence so long as they function to direct the expression of the coding sequence. Thus, for example, intervening untranslated yet transcribed sequences can be present between a promoter sequence and the coding sequence and the promoter sequence can still be considered “operably linked” to the coding sequence. In fact, such sequences need not reside on the same contiguous DNA molecule (i.e. chromosome) and may still have interactions resulting in altered regulation.

A “PAM mutation” refers to one or more edits to a target sequence that removes, mutates, or otherwise renders inactive a PAM or spacer region in the target sequence.

As used herein, the terms “protein” and “peptide” and “polypeptide” are used interchangeably. Proteins may or may not be made up entirely of amino acids.

A “promoter” or “promoter sequence” is a DNA regulatory region capable of binding RNA polymerase and initiating transcription of a polynucleotide or polypeptide coding sequence such as messenger RNA, ribosomal RNA, small nuclear or nucleolar RNA, guide RNA, or any kind of RNA transcribed by any class of any RNA polymerase I, II or III. Promoters may be constitutive or inducible.

As used herein, the terms “repair template” or “homology arm” refer to 1) nucleic acid that is designed to facilitate introduction of a DNA sequence modification (insertion, deletion, substitution) into a locus by homologous recombination using nucleic acid-guided nucleases, or 2) a nucleic acid that serves as a template (including a desired edit) to be incorporated into target DNA by reverse transcriptase in a CREATE fusion editing (CFE) system. For homology-directed repair, a repair template or homology arm may have sufficient homology to the regions flanking the “cut site” or the site to be edited in the genomic target sequence. For template-directed repair, the repair template or homology arm has homology to the genomic target sequence except at the position of the desired edit although synonymous edits may be present in the homologous (e.g., non-edit) regions. The length of the repair template(s) or homology arm(s) will depend on, e.g., the type and size of the modification being made. In many instances and preferably, the repair template will have two regions of sequence homology (e.g., two homology arms) complementary to the genomic target locus flanking the locus of the desired edit in the genomic target locus. Typically, an “edit region” or “edit locus” or “DNA sequence modification” region—the nucleic acid modification that one desires to be introduced into a genome target locus in a cell (e.g., the desired edit)—will be located between two regions of homology. The DNA sequence modification may change one or more bases of the target genomic DNA sequence at one specific site or multiple specific sites. A change may include changing 1, 2, 3, 4, 5, 10, 15, 20, 25, 30, 35, 40, 50, 75, 100, 150, 200, 300, 400, or 500 or more base pairs of the target sequence. A deletion or insertion may be a deletion or insertion of 1, 2, 3, 4, 5, 10, 15, 20, 25, 30, 40, 50, 75, 100, 150, 200, 300, 400, or 500 or more base pairs of the target sequence.

As used herein the term “selectable marker” refers to a gene introduced into a cell, which confers a trait suitable for artificial selection. General use selectable markers are well known to those of ordinary skill in the art. For examples, selectable markers can use means that deplete a cell population to enrich for editing, and include ampicillin/carbenicillin, kanamycin, chloramphenicol, nourseothricin N-acetyl transferase, erythromycin, tetracycline, gentamicin, bleomycin, streptomycin, puromycin, hygromycin, blasticidin, and G418 or other selectable markers may be employed. In addition, selectable markers include physical markers that confer a phenotype that can be utilized for physical or computations cell enrichment, e.g., optical selectable markers such as fluorescent proteins (e.g., green fluorescent protein, blue fluorescent protein) and cell surface handles.

The term “specifically binds” as used herein includes an interaction between two molecules, e.g., an engineered peptide antigen and a binding target, with a binding affinity represented by a dissociation constant of about 10⁻⁷M, about 10⁻⁸M, about 10⁻⁹ M, about 10⁻¹⁰ M, about 10⁻¹¹M, about 10⁻¹²M, about 10⁻¹³M, about 10⁻¹⁴M or about 10⁻¹⁵M.

The terms “target genomic DNA sequence”, “cellular target sequence”, “target sequence”, or “genomic target locus” refer to any locus in vitro or in vivo, or in a nucleic acid (e.g., genome or episome) of a cell or population of cells, in which a change of at least one nucleotide is desired using a nucleic acid-guided nuclease editing system. The target sequence can be a genomic locus or extrachromosomal locus.

The terms “transformation”, “transfection” and “transduction” refer to one or more processes of introducing exogenous DNA into cells.

The term “variant” may refer to a polypeptide or polynucleotide that differs from a reference polypeptide or polynucleotide but retains essential properties. A typical variant of a polypeptide differs in amino acid sequence from another reference polypeptide. Generally, differences are limited so that the sequences of the reference polypeptide and the variant are closely similar overall and, in many regions, identical. A variant and reference polypeptide may differ in amino acid sequence by one or more modifications (e.g., substitutions, additions, and/or deletions). A variant of a polypeptide may be a conservatively modified variant. A substituted or inserted amino acid residue may or may not be one encoded by the genetic code (e.g., a non-natural amino acid). A variant of a polypeptide may be naturally occurring, such as an allelic variant, or it may be a variant that is not known to occur naturally.

A “vector” is any of a variety of nucleic acids that comprise a desired sequence or sequences to be delivered to and/or expressed in a cell. Vectors are typically composed of DNA, although RNA vectors are also available. Vectors include, but are not limited to, plasmids, fosmids, phagemids, virus genomes, synthetic chromosomes, and the like. In the present disclosure, the term “editing vector” includes a coding sequence for a nuclease, a gRNA sequence to be transcribed, and a donor DNA sequence. In other embodiments, however, two vectors—an engine vector comprising the coding sequence for a nuclease, and an editing vector, comprising the gRNA sequence to be transcribed and the donor DNA sequence—may be used.

### BRIEF DESCRIPTION OF THE DRAWINGS

The foregoing and other features and advantages of the present invention will be more fully understood from the following detailed description of illustrative embodiments taken in conjunction with the accompanying drawings in which:

FIGS. 1A-1D depict an automated multi-module instrument and components thereof with which to practice the recursive editing methods as taught herein, according to certain embodiments.

FIG. 2A depicts a rotating growth vial for use with the cell growth module described herein, according to certain embodiments. FIG. 2B illustrates a perspective view of a rotating growth vial in a cell growth module, according to certain embodiments. FIG. 2C depicts a cut-away view of the cell growth module from FIG. 2B, according to certain embodiments. FIG. 2D illustrates the cell growth module of FIG. 2B coupled to LED, detector, and temperature regulating components, according to certain embodiments.

FIG. 3A is an example model of tangential flow filtration used in the TFF device presented herein, according to certain embodiments. FIG. 3B depicts a top view of a lower member of an exemplary TFF device, according to certain embodiments. FIG. 3C depicts a top view of upper and lower members and a membrane of an exemplary TFF device, according to certain embodiments. FIG. 3D depicts a bottom view of upper and lower members and a membrane of an exemplary TFF device, according to certain embodiments. FIGS. 3E-3K depict various views of a TFF module having fluidically coupled reservoirs, according to certain embodiments. FIG. 3L is an exemplary pneumatic architecture diagram for the TFF module described in relation to FIGS. 3E-3K, according to certain embodiments.

FIG. 4A shows an exemplary flow-through electroporation device (here, there are six such devices co-joined), according to certain embodiments. FIG. 4B is a top view of an exemplary flow-through electroporation device, according to certain embodiments. FIG. 4C depicts a top view of a cross section of the electroporation device of FIG. 4C, according to certain embodiments. FIG. 4D is a side view cross section of a lower portion of the electroporation devices of FIGS. 4C and 4D, according to certain embodiments.

FIGS. 5A and 5B depict the structure and components of a reagent cartridge, according to certain embodiments.

FIG. 6 is a simplified block diagram of an exemplary automated multi-module cell processing instrument, according to certain embodiments.

FIG. 7A depicts an exemplary engine vector for creating edits, and FIG. 7B depicts an exemplary editing vector for creating edits, according to certain embodiments.

FIG. 8 depicts a flow diagram of an exemplary process for producing and screening for an antibiotic compound, according to certain embodiments.

FIGS. 9A and 9B schematically depict operations of the process of FIG. 8, according to embodiments described herein. FIGS. 9C and 9D graphically depict operations of the process of FIG. 8, according to embodiments described herein.

FIG. 10 depicts a flow diagram of an exemplary process for producing and screening for an antibiotic compound, according to certain embodiments.

FIGS. 11A and 11B schematically depict operations of the process of FIG. 10, according to embodiments described herein.

### THE INVENTION IN GENERAL

This disclosure is directed to the editing of organisms for enhanced biosynthesis and rapid screening of novel antibiotic compounds. Bacteria have evolved resistance to many current antibiotics, often by alterations in the molecular target site. This resistance is often attributed to the overuse and misuse of antibiotics, as well as a lack of new drug development in recent years. Thus, there is a growing need to produce novel and potent antibiotics with new molecular target sites and modes of action that overcome widespread antibiotic resistance.

Currently, antibiotic development and screening is an onerous, multistep process that requires a great deal of time and resources. The present disclosure provides devices, instruments, and methods, including automated methods, for enhanced production and efficient screening of such biosynthesized antibiotics. More particularly, the present disclosure provides for accelerated biosynthesis and screening at a single-cell scale.

### Nuclease-Directed Genome Editing Herein, Generally

The compositions and methods described herein are employed to perform nuclease-directed genome editing to introduce desired edits to a population of live microbial cells. In some embodiments, a single edit is introduced in a single round of editing. In some embodiments, multiple edits are introduced in a single round of editing using simultaneous editing, e.g., the introduction of two or more edits on a single vector (combinatorial editing) or multiple vector (multiplex editing). In some embodiments, recursive cell editing is performed where edits are introduced in successive rounds of editing.

A nucleic acid-guided nuclease, or CREATE fusion enzyme, complexed with an appropriate synthetic guide nucleic acid in a cell can cut the genome of the cell at a desired location. The guide nucleic acid helps the nucleic acid-guided nuclease recognize and cut the DNA at a specific target sequence (either a cellular target sequence or a curing target sequence). By manipulating the nucleotide sequence of the guide nucleic acid, the nucleic acid-guided nuclease may be programmed to target any DNA sequence for cleavage as long as an appropriate protospacer adjacent motif (PAM) is nearby. In certain aspects, the nucleic acid-guided nuclease editing system may use two separate guide nucleic acid molecules that combine to function as a guide nucleic acid, e.g., a CRISPR RNA (crRNA) and trans-activating CRISPR RNA (tracrRNA). In other aspects and preferably, the guide nucleic acid is a single guide nucleic acid construct that includes both 1) a guide sequence capable of hybridizing to a genomic target locus, and 2) a scaffold sequence capable of interacting or complexing with a nucleic acid-guided nuclease.

In general, a guide nucleic acid (e.g., a gRNA or CFgRNA) complexes with a compatible nucleic acid-guided nuclease or CREATE fusion enzyme and can then hybridize with a target sequence, thereby directing the nuclease to the target sequence. A guide nucleic acid can be DNA or RNA; alternatively, a guide nucleic acid may comprise both DNA and RNA. In some embodiments, a guide nucleic acid may comprise modified or non-naturally occurring nucleotides. In cases where the guide nucleic acid comprises RNA, the gRNA may be encoded by a DNA sequence on a polynucleotide molecule such as a plasmid, linear construct, or the coding sequence may and preferably does reside within an editing cassette. Methods and compositions for designing and synthesizing editing cassettes and libraries of editing cassettes are described in U.S. Pat. Nos. 10,240,167; 10,266,849; 9,982,278; 10,351,877; 10,364,442; 10,435,715; 10,465,207; 10,669,559; 10,711,284; 10,731,180; and 11,078,498; all of which are incorporated by reference herein.

A guide nucleic acid comprises a guide sequence, where the guide sequence is a polynucleotide sequence having sufficient complementarity with a target sequence to hybridize with the target sequence and direct sequence-specific binding of a complexed nucleic acid-guided nuclease to the target sequence. The degree of complementarity between a guide sequence and the corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences. In some embodiments, a guide sequence is about or more than about 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20 nucleotides in length. Preferably the guide sequence is 10-30 or 15-20 nucleotides long, or 15, 16, 17, 18, 19, or 20 nucleotides in length.

In certain embodiments of the present methods and compositions, the guide nucleic acids are provided as a sequence to be expressed from a plasmid or vector and comprises both the guide sequence and the scaffold sequence as a single transcript under the control of a promoter, e.g., an inducible or constitutive promoter. In certain embodiments, the guide nucleic acid may be part of an editing cassette that encodes a repair template for effecting an edit in the cellular target sequence, and/or one or more homology arms. Alternatively, the guide nucleic acid may not be part of the editing cassette and instead may be encoded on the vector backbone, such as an editing plasmid backbone. For example, a sequence coding for a guide nucleic acid can be assembled or inserted into an editing vector backbone first, followed by insertion of the repair template in, e.g., an editing cassette. In other cases, the repair template in, e.g., an editing cassette can be inserted or assembled into an editing vector backbone first, followed by insertion of the sequence coding for the guide nucleic acid. In certain embodiments, the sequence encoding the guide nucleic acid and repair template are located together in a rationally designed editing cassette and are simultaneously inserted or assembled via gap repair into a linear vector or backbone to create an editing vector.

The guide nucleic acids are engineered to target a desired target sequence (e.g., a cellular “editing” target sequence) by altering the guide sequence so that the guide sequence is complementary to a desired target sequence, thereby allowing hybridization between the guide sequence and the target sequence. The target sequence can be any polynucleotide endogenous or exogenous to a cell, or in vitro. In certain embodiments, a target sequence can be a sequence encoding a gene product (e.g., a protein, a non-coding sequence (e.g., a regulatory polynucleotide, an intron, a proto-spacer adjacent motif (PAM) sequence, or “junk” DNA).

In general, to generate an edit in the target sequence, a gRNA/nuclease complex binds to the target sequence as determined by the guide RNA, and the nuclease or CF enzyme recognizes a PAM sequence adjacent to or in proximity to the target sequence. The precise preferred PAM sequence and length requirements for different nucleic acid-guided nucleases vary; however, PAMs typically are 2-10 or so base-pairs in length and, depending on the nuclease, can be 5′ or 3′ to the target sequence. Engineering of the PAM-interacting domain of a nucleic acid-guided nuclease may allow for alteration of PAM specificity, improve target site recognition fidelity, decrease target site recognition fidelity, or increase the versatility of a nucleic acid-guided nuclease.

In most embodiments, genome editing of a cellular target sequence both introduces a desired DNA change to a cellular target sequence (an “intended” edit), e.g., the genomic DNA of a cell, and removes, mutates, or renders inactive a PAM region in the cellular target sequence (an “immunizing edit”), thereby rendering the target site immune to further nuclease binding. Rendering the PAM at the cellular target sequence inactive precludes additional editing of the cell genome at that cellular target sequence, e.g., upon subsequent exposure to a nucleic acid-guided nuclease complexed with a synthetic guide nucleic acid in later rounds of editing. Thus, cells having the desired cellular target sequence edit and an altered PAM can be selected for by using a nucleic acid-guided nuclease complexed with a synthetic guide nucleic acid complementary to the cellular target sequence. Cells that did not undergo the first editing event may be cut rendering a double-stranded DNA break, and thus will not continue to be viable. The cells containing the desired cellular target sequence edit and PAM alteration will not be cut, as these edited cells no longer contain the necessary PAM site and will continue to grow and propagate.

As for the nuclease or CF enzyme component of the nucleic acid-guided nuclease or CF enzyme, a polynucleotide sequence encoding the nucleic acid-guided nuclease or CF enzyme can be codon optimized for expression in particular cell types, such as bacterial, yeast, and, here, mammalian cells. The choice of the nucleic acid-guided nuclease or CF enzyme to be employed depends on many factors, such as what type of edit is to be made in the target sequence and whether an appropriate PAM is located close to the desired target sequence. Nucleases of use in the methods described herein include but are not limited to Cas9, Cas12, MAD2, or MAD7, MAD2007 or other MADzymes and MADzyme systems (see U.S. Pat. Nos. 10,604,746; 10,655,114; 10,649,754; 10,876,102; 10,833,077; 11,053,485; 10,704,022; 10,745,678; 10,724,021; 10,767,169; 10,870,761; 10,011,849; 10,435,714; 10,626,416; 9,982,279; and 10,337,028; and U.S. Ser. Nos. 16/953,253; 17/374,628; 17,200,074; 17,200,089; 17/200,110; 16/953,233; 17/463,498; 63/134,938; 16/819,896; 17/179,193; and 16/421,783 for sequences and other details related to engineered and naturally-occuring MADzymes). CF enzymes typically comprise a CRISPR nucleic acid-guided nuclease engineered to cut one DNA strand in the target DNA rather than making a double-stranded cut, and the nickase portion is fused to a reverse transcriptase. In specific aspects, the one or more nickases include MAD7 nickase, MAD2001 nickase, MAD2007 nickase, MAD2008 nickase, MAD2009 nickase, MAD2011 nickase, MAD2017 nickase, MAD2019 nickase, MAD297 nickase, MAD298 nickase, MAD299 nickase, or other MAD-series nickases, variants thereof, and/or combinations thereof as described in U.S. Pat. Nos. 10,883,077; 11,053,485; 11,085,030; 11,200,089; 11,193,115; and U.S. Ser. No. 17/463,498. A coding sequence for a desired nuclease or CF enzyme may be on an “engine vector” along with other desired sequences such as a selective marker(s), or a coding sequence for the desired nuclease or nickase may reside on the editing vector or may be transfected into a cell as a protein.

In some embodiments, the nuclease or CF enzyme may be under the transcriptional control of a promoter. The promoter may be separate from but the same as the promoter controlling transcription of the guide nucleic acid; that is, a separate promoter drives the transcription of the nuclease and guide nucleic acid sequences but the two promoters may be the same type of promoter (e.g., both are inducible pL promoters). Alternatively, the promoter controlling expression of the nuclease or CF enzyme may be different from the promoter controlling transcription of the guide nucleic acid; that is, e.g., the nuclease may be under the control of, e.g., a pTEF promoter, and the guide nucleic acid may be under the control of, e.g., a pCYC1 promoter. In certain embodiments, the promoter controlling expression of the nuclease and/or the promoter controlling expression of the guide nucleic acid is an inducible promoter; for example, the nuclease may be under the control of, e.g., a pBAD inducible promoter, and the guide nucleic acid may be under the control of, e.g., a pL inducible promoter.

Another component of the nucleic acid-guided nuclease system or CF system is the donor nucleic acid or repair template comprising homology to the cellular target sequence. A repair template typically is designed to serve as a template for homologous recombination with a cellular target sequence cleaved by the nucleic acid-guided nuclease, or the repair template serves as the template for template-directed repair via the CF enzyme, as a part of the gRNA/nuclease complex. For the present methods and compositions, the repair template typically is on the same vector and, in certain embodiments, in the same editing cassette, as a guide nucleic acid for editing, and may be under the control of the same promoter as the editing gRNA (that is, a single promoter driving the transcription of both the editing gRNA and the repair template). A repair template polynucleotide may be of any suitable length, such as about or more than about 20, 25, 50, 75, 100, or more nucleotides in length. In certain preferred aspects, the repair template can be provided as an oligonucleotide of between 20-100 nucleotides, such as between 30-75 nucleotides. When optimally aligned, the repair template overlaps with (is complementary to) the cellular target sequence by, e.g., about 20, 25, 30, 35, 40, 50, 60, 70, 80, 90 or more nucleotides.

The repair template generally comprises two regions that are complementary to a portion of the cellular target sequence (e.g., homology arms). In certain embodiments of the present methods and compositions, the two homology arms flank an intended edit, e.g., at least one alteration as compared to the cellular target sequence, such as a DNA sequence insertion, which may be part of the repair template. In certain embodiments, the repair template comprises two homology arms that do not flank the intended edit. In such embodiments, the homology arms may be encoded on an editing vector backbone, or in an editing cassette with the edit.

An editing cassette may further comprise one or more primer binding sites. The primer binding sites are used to amplify the editing cassette by using oligonucleotide primers as described infra and may be biotinylated or otherwise labeled.

In addition, the editing cassette may comprise a barcode. A barcode is a unique DNA sequence that corresponds to the donor DNA sequence such that the barcode can identify the edit made to the corresponding cellular target sequence. The barcode typically comprises four or more nucleotides. In some embodiments, the editing cassettes comprise a collection or library of editing gRNAs and of donor nucleic acids representing, e.g., gene-wide or genome-wide libraries of editing gRNAs and donor nucleic acids. The library of editing cassettes is cloned into vector backbones where, e.g., each different donor nucleic acid is associated with a different barcode.

In certain embodiments, the plasmid and/or vector encoding components of the nucleic acid-guided nuclease system, e.g., the editing vector, further encodes a nucleic acid-guided nuclease comprising one or more nuclear localization sequences (NLSs), such as about or more than about 1, 2, 3, 4, 5, 6, 7, 8, 9, 10, or more NLSs, particularly as an element of the nuclease sequence. In some embodiments, the engineered nuclease comprises NLSs at or near the amino-terminus, NLSs at or near the carboxy-terminus, or a combination.

### Automated Cell Editing Instruments and Modules to Perform Nucleic Acid-Guided Nuclease Editing

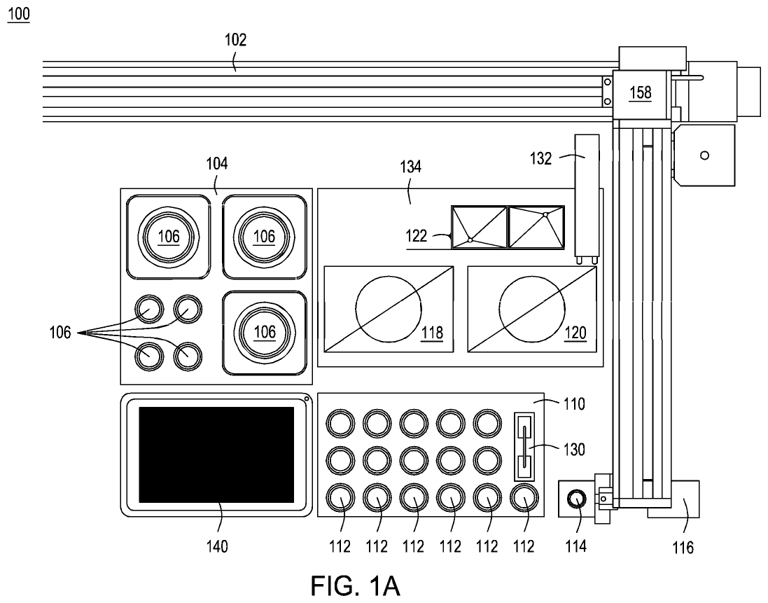

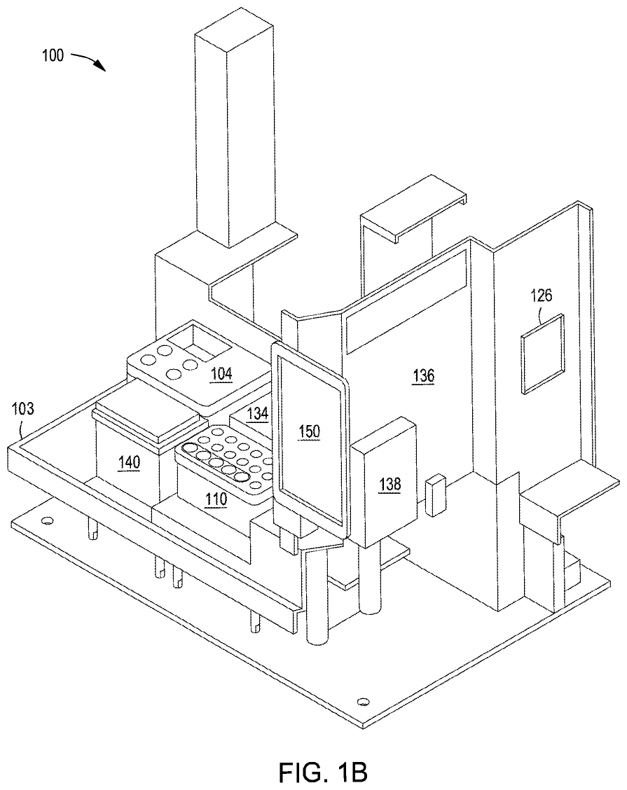

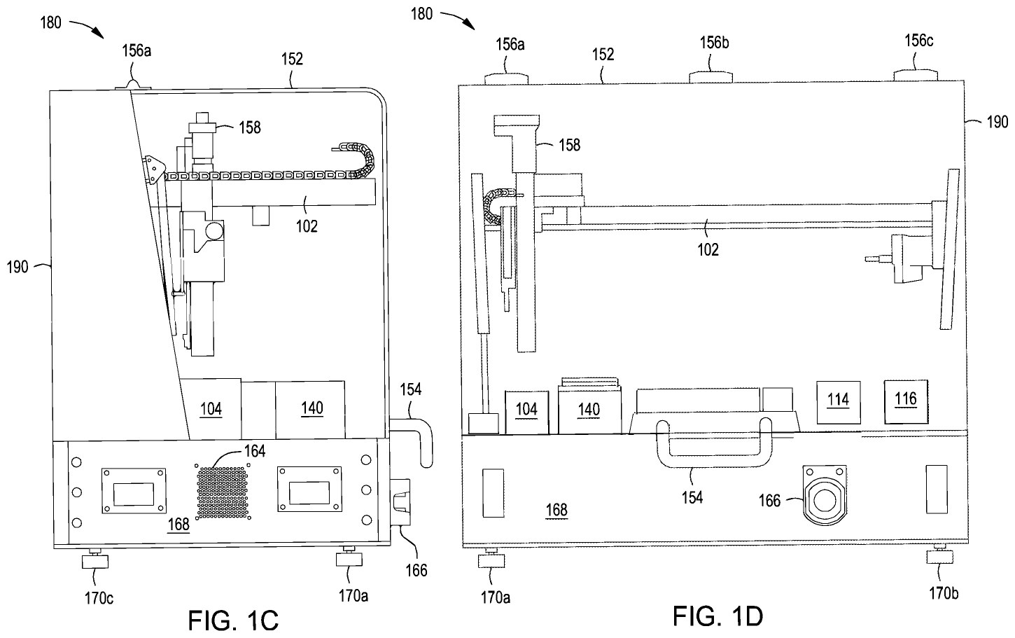

FIG. 1A depicts an exemplary automated multi-module cell processing instrument 100 to, e.g., perform one of the exemplary workflows described herein. The instrument 100, for example, may be and preferably is designed as a desktop instrument for use within a laboratory environment. The instrument 100 may incorporate a mixture of reusable and disposable elements for performing various staged processes in conducting automated genome cleavage and/or editing in cells. Illustrated is a gantry 102, providing an automated mechanical motion system (actuator) (not shown) that supplies XYZ axis motion control to, e.g., an automated liquid handling system 158 including, e.g., an air displacement pipette as well as modules of the automated multi-module cell processing instrument 100. In some automated multi-module cell processing instruments, the air displacement pipettor 132 is moved by gantry 102 and the various modules and reagent cartridges remain stationary; however, in other embodiments, the liquid handling system may stay stationary while the various modules are moved. Also included in the automated multi-module cell processing instrument 100 is reagent cartridge 110 comprising reservoirs 112 and transformation module 130, as well as a wash cartridge 104 comprising reservoirs 106. The wash cartridge 104 may be configured to accommodate large tubes, for example, wash solutions, or solutions that are used often throughout an iterative process. In one example, wash cartridge 104 may be configured to remain in place when two or more reagent cartridges 110 are sequentially used and replaced. Although reagent cartridge 110 and wash cartridge 104 are shown in FIG. 1A as separate cartridges, the contents of wash cartridge 104 may be incorporated into reagent cartridge 110. Note in this embodiment transformation module 130 is contained within reagent cartridge 110; however, in alternative embodiments transformation module 130 is contained within its own module or may be part of another module, such as a growth module.

The wash and reagent cartridges 104 and 110 in some implementations, are disposable kits provided for use in the automated multi-module cell editing instrument 100. For example, a user may open and position each of the reagent cartridge 110 and the wash cartridge 104 within a chassis of the automated multi-module cell editing instrument prior to activating cell processing.

Also illustrated is the robotic handling system 158 including the gantry 102 and air displacement pipettor 132. In some examples, the robotic handling system 158 may include an automated liquid handling system such as those manufactured by Tecan Group Ltd. of Mannedorf, Switzerland, Hamilton Company of Reno, Nev. (see, e.g., WO2018015544A1), or Beckman Coulter, Inc. of Fort Collins, Colo. (see, e.g., US20160018427A1). Pipette tips may be provided in a pipette transfer tip supply (not shown) for use with the air displacement pipettor 132.

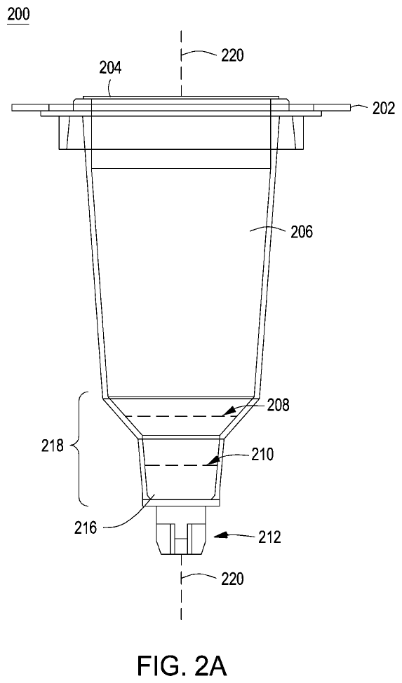

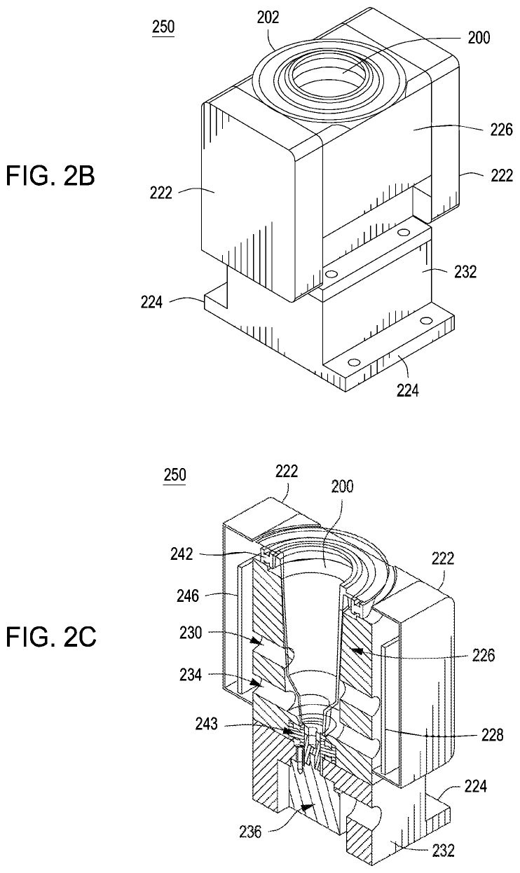

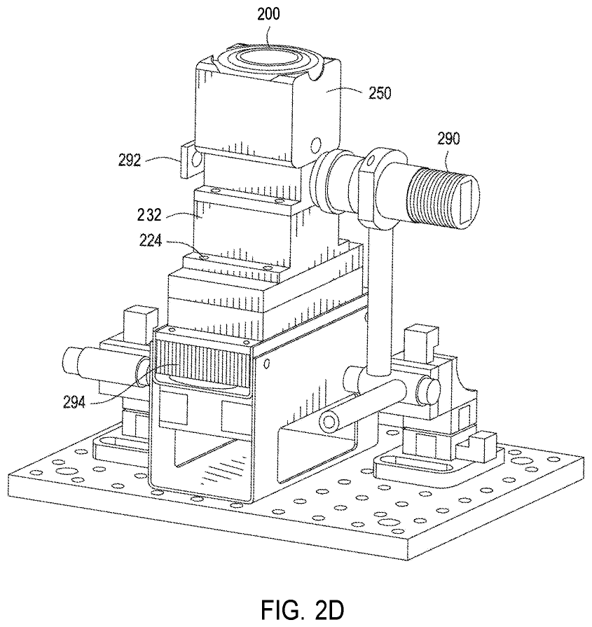

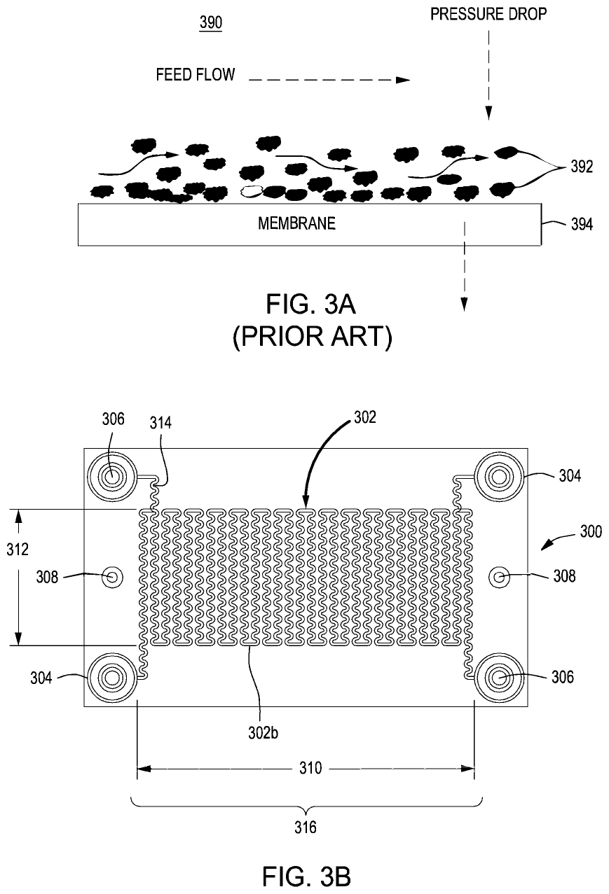

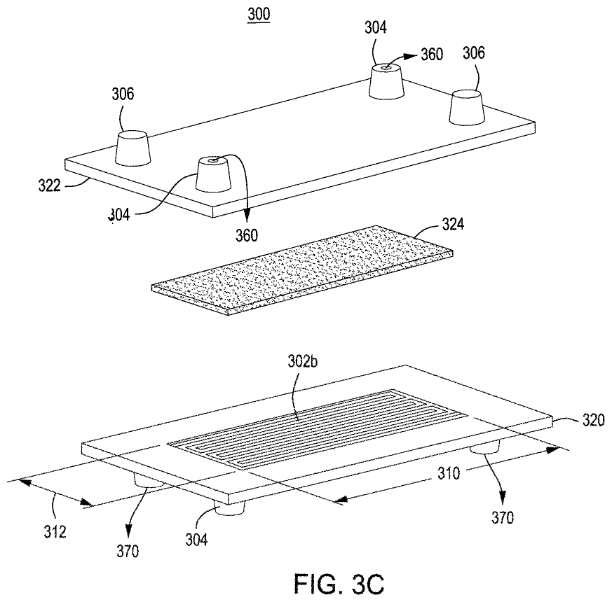

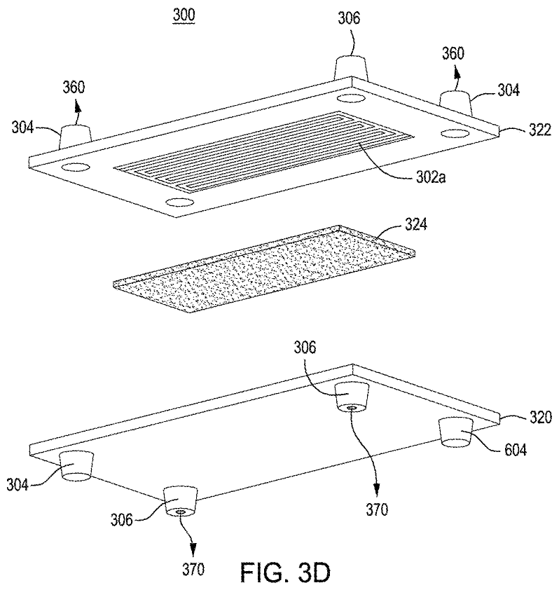

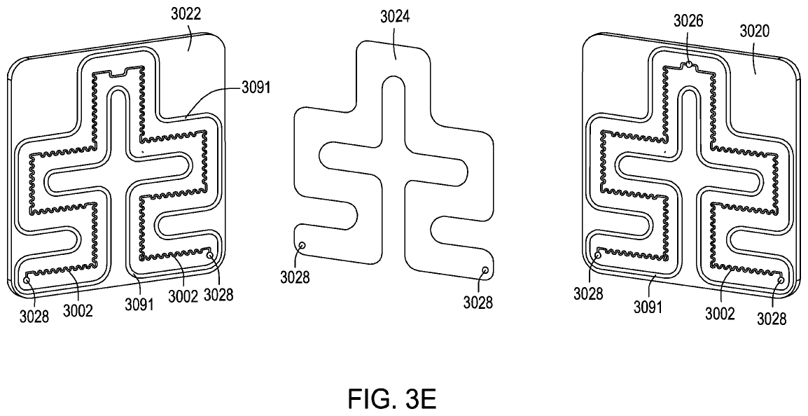

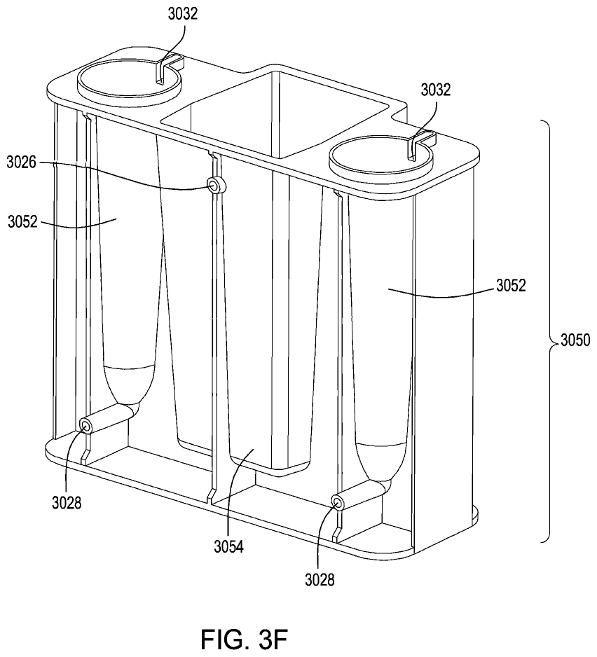

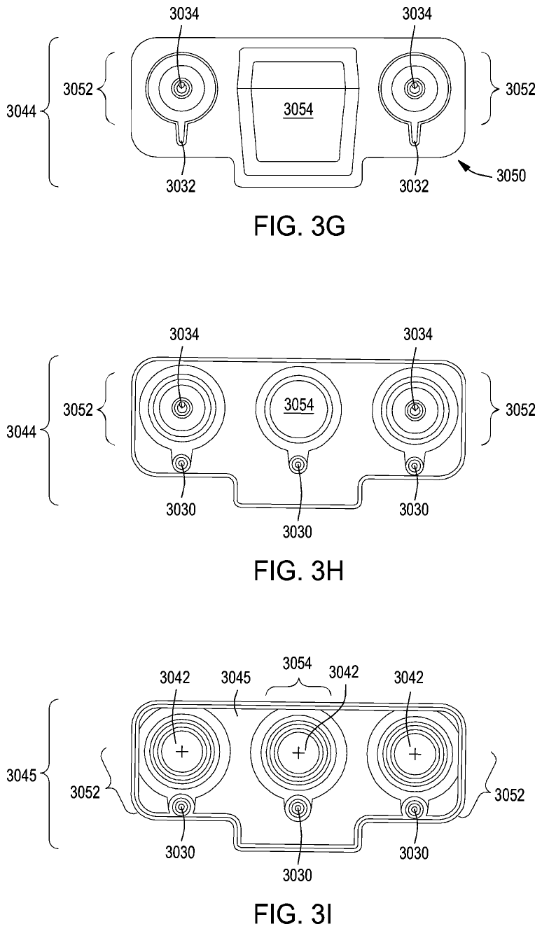

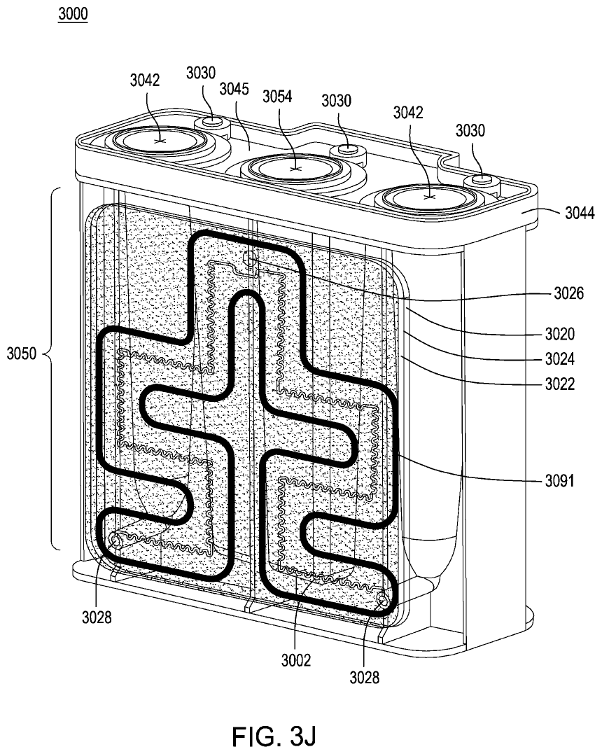

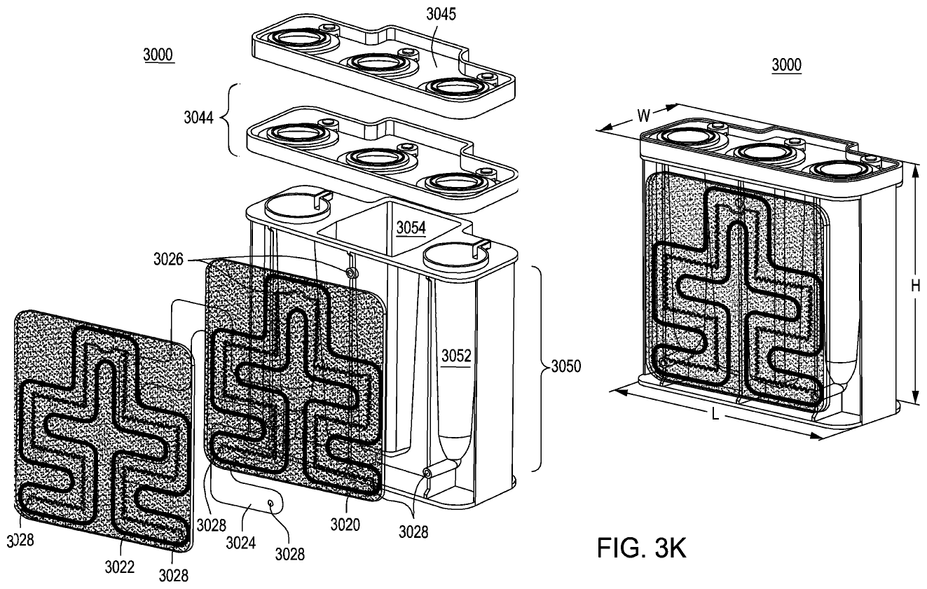

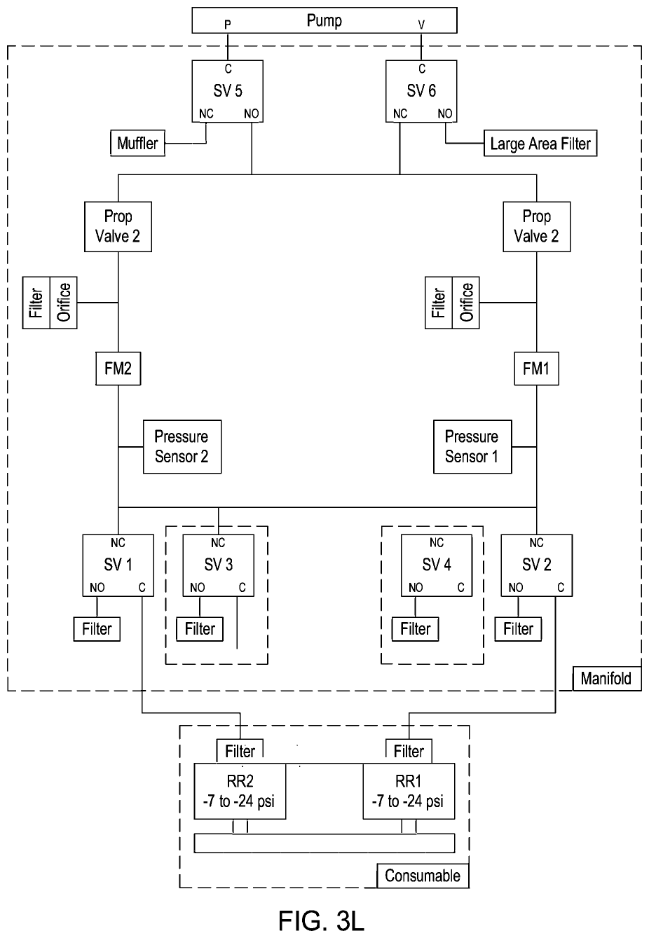

Components of the cartridges 104, 110, in some implementations, are marked with machine-readable indicia (not shown), such as bar codes, for recognition by the robotic handling system 158. For example, the robotic handling system 158 may scan containers within each of the cartridges 104, 110 to confirm contents. In other implementations, machine-readable indicia may be marked upon each cartridge 104, 110, and the processing system 126 (shown in FIG. 1D) of the automated multi-module cell editing instrument 100 may identify a stored materials map based upon the machine-readable indicia. The exemplary automated multi-module cell processing instrument 100 of FIG. 1A further comprises a cell growth module 134. (Note, all modules recited briefly here are described in detail below.) In the embodiment illustrated in FIG. 1A, the cell growth module 134 comprises two cell growth vials 118, 120 (described in greater detail below in relation to FIGS. 2A-2D) as well as a cell concentration module 122 (described in detail in relation to FIGS. 3A-3F). In alternative embodiments, the cell concentration module 122 may be separate from cell growth module 134, e.g., in a separate, dedicated module. Also illustrated as part of the automated multi-module cell processing instrument 100 of FIG. 1A is an optional enrichment module 140, served by, e.g., robotic handling system 158 and air displacement pipettor 132. Also seen are an optional nucleic acid assembly/desalting module 114 comprising a reaction chamber or tube receptacle (not shown) and a magnet 116 to allow for purification of nucleic acids using, e.g., magnetic solid phase reversible immobilization (SPRI) beads (Applied Biological Materials Inc., Richmond, BC). The cell growth module, cell concentration module, transformation module, enrichment module, and reagent cartridge are described in greater detail below.

FIG. 1B is a plan view of the front of the exemplary multi-module cell processing instrument 100 depicted in FIG. 1A. Cartridge-based source materials (such as in reagent cartridge 110), for example, may be positioned in designated areas on a deck 103 of the instrument 100 for access by a robotic handling instrument (not shown in this figure). As illustrated in FIG. 1B, the deck 103 may include a protection sink such that contaminants spilling, dripping, or overflowing from any of the modules of the instrument 100 are contained within a lip of the protection sink. In addition to reagent cartridge 110, also seen in FIG. 1B is wash cartridge 104, optional enrichment module 140, and a portion of growth module 134. Also seen in this view is touch screen display 150, transformation module controls 138, electronics rack 136, and processing system 126.

FIGS. 1C through 1D illustrate multi-module cell processing instruments 180 comprising chassis 190 for use in desktop versions the cell editing instrument 180. For example, the chassis 190 may have a width of about 24-48 inches, a height of about 24-48 inches and a depth of about 24-48 inches. Chassis 190 may be and preferably is designed to hold multiple modules and disposable supplies used in automated cell processing. Further, chassis 190 may mount a robotic handling system 158 for moving materials between modules.

As illustrated, the chassis 190 includes a cover 152 having a handle 154 and hinges 156a-156c for lifting the cover and accessing the interior of the chassis 190. A cooling grate 164 allows for air flow via an internal fan (not shown). Further, the chassis 190 is lifted by adjustable feet 170 (feet 170 a-c are shown). The feet 170a-170c, for example, may provide additional air flow beneath the chassis 190. A control button 166, in some embodiments, allows for single-button automated start and/or stop of cell processing within the chassis 190.

Inside the chassis 190, in some implementations, a robotic handling system 158 is disposed along a gantry 102 above materials cartridges 104 and 110. Control circuitry, liquid handling tubes, air pump controls, valves, thermal units (e.g., heating and cooling units) and other control mechanisms, in some embodiments, are disposed below a deck of the chassis 190, in a control box region 168. Also seen in FIG. 1D is enrichment module 140 and nucleic acid assembly module 114 comprising a magnet 116

Although not illustrated, in some embodiments a display screen may be positioned on the front face of the chassis 190, for example covering a portion of the cover (e.g., see FIG. 1B). The display screen may provide information to the user regarding the processing status of the automated multi-module cell editing instrument. In another example, the display screen may accept inputs from the user for conducting the cell processing.

### The Rotating Cell Growth Module

FIG. 2A shows one embodiment of a rotating growth vial 200 for use with the cell growth device described herein configured to grow all cell types including cells lines and primary cells. The rotating growth vial is an optically-transparent container having an open end 204 for receiving liquid media and cells, a central vial region 206 that defines the primary container for growing cells, a tapered-to-constricted region 218 defining at least one light path 210, a closed end 216, and a drive engagement mechanism 212. The rotating growth vial has a central longitudinal axis 220 around which the vial rotates, and the light path 210 is generally perpendicular to the longitudinal axis of the vial. The first light path 210 is positioned in the lower constricted portion of the tapered-to-constricted region 218. Optionally, some embodiments of the rotating growth vial 200 have a second light path 208 in the tapered region of the tapered-to-constricted region 218. Both light paths in this embodiment are positioned in a region of the rotating growth vial that is constantly filled with the cell culture (cells+growth media) and is not affected by the rotational speed of the growth vial. The first light path 210 is shorter than the second light path 208 allowing for sensitive measurement of OD values when the OD values of the cell culture in the vial are at a high level (e.g., later in the cell growth process), whereas the second light path 208 allows for sensitive measurement of OD values when the OD values of the cell culture in the vial are at a lower level (e.g., earlier in the cell growth process). Also shown is lip 202, which allows the rotating growth vial to be seated in a growth module (not shown) and further allows for easy handling for the user.

In some configurations of the rotating growth vial, the rotating growth vial has two or more “paddles” or interior features disposed within the rotating growth vial, extending from the inner wall of the rotating growth vial toward the center of the central vial region. In some aspects, the width of the paddles or features varies with the size or volume of the rotating growth vial, and may range from 1/20 to just over ⅓ the diameter of the rotating growth vial, or from 1/15 to ¼ the diameter of the rotating growth vial, or from 1/10 to ⅕ the diameter of the rotating growth vial. In some aspects, the length of the paddles varies with the size or volume of the rotating growth vial, and may range from ⅘ to ¼ the length of the main body of the rotating growth vial, or from ¾ to ⅓ the length of the main body of the rotating growth vial, or from ½ to ⅓ the length of the main body of the rotating growth vial. In other aspects, there may be concentric rows of raised features disposed on the inner surface of the main body of the rotating growth vial arranged horizontally or vertically; and in other aspects, there may be a spiral configuration of raised features disposed on the inner surface of the main body of the rotating growth vial. In alternative aspects, the concentric rows of raised features or spiral configuration may be disposed upon a post or center structure of the rotating growth vial. Though described above as having two paddles, the rotating growth vial may comprise 3, 4, 5, 6 or more paddles, and up to 20 paddles. The number of paddles will depend upon, e.g., the size or volume of the rotating growth vial. The paddles may be arranged symmetrically as single paddles extending from the inner wall of the vial into the interior of the vial, or the paddles may be symmetrically arranged in groups of 2, 3, 4 or more paddles in a group (for example, a pair of paddles opposite another pair of paddles) extending from the inner wall of the vial into the interior of the vial. In another embodiment, the paddles may extend from the middle of the rotating growth vial out toward the wall of the rotating growth vial, from, e.g., a post or other support structure in the interior of the rotating growth vial.

The drive engagement mechanism 212 engages with a motor (not shown) to rotate the vial. In some embodiments, the motor drives the drive engagement mechanism 212 such that the rotating growth vial is rotated in one direction only, and in other embodiments, the rotating growth vial is rotated in a first direction for a first amount of time or periodicity, rotated in a second direction (i.e., the opposite direction) for a second amount of time or periodicity, and this process may be repeated so that the rotating growth vial (and the cell culture contents) are subjected to an oscillating motion. The first amount of time and the second amount of time may be the same or may be different. The amount of time may be 1, 2, 3, 4, 5, or more seconds, or may be 1, 2, 3, 4 or more minutes. In another embodiment, in an early stage of cell growth the rotating growth vial may be oscillated at a first periodicity (e.g., every 60 seconds), and then a later stage of cell growth the rotating growth vial may be oscillated at a second periodicity (e.g., every one second) different from the first periodicity.

The rotating growth vial 200 may be reusable or, preferably, the rotating growth vial is consumable. In some embodiments, the rotating growth vial is consumable and is presented to the user pre-filled with growth medium, where the vial is hermetically sealed at the open end 204 with a foil seal. A medium-filled rotating growth vial packaged in such a manner may be part of a kit for use with a stand-alone cell growth device or with a cell growth module that is part of an automated multi-module cell processing instrument. To introduce cells into the vial, a user need only pipette up a desired volume of cells and use the pipette tip to punch through the foil seal of the vial. Open end 204 may optionally include an extended lip 202 to overlap and engage with the cell growth device (not shown). In automated systems, the rotating growth vial 200 may be tagged with a barcode or other identifying means that can be read by a scanner or camera that is part of the automated system (not shown).

The volume of the rotating growth vial 200 and the volume of the cell culture (including growth medium) may vary greatly, but the volume of the rotating growth vial 200 must be large enough for the cell culture in the growth vial to get proper aeration while the vial is rotating. In practice, the volume of the rotating growth vial 200 may range from 1-250 ml, 2-100 ml, from 5-80 ml, 10-50 ml, or from 12-35 ml. Likewise, the volume of the cell culture (cells+growth media) should be appropriate to allow proper aeration in the rotating growth vial. Thus, the volume of the cell culture should be approximately 10-85% of the volume of the growth vial or from 20-60% of the volume of the growth vial. For example, for a 35 ml growth vial, the volume of the cell culture would be from about 4 ml to about 27 ml, or from 7 ml to about 21 ml.

The rotating growth vial 200 preferably is fabricated from a bio-compatible optically transparent material—or at least the portion of the vial comprising the light path(s) is transparent. Additionally, material from which the rotating growth vial is fabricated should be able to be cooled to about 4° C. or lower and heated to about 55° C. or higher to accommodate both temperature-based cell assays and long-term storage at low temperatures. Further, the material that is used to fabricate the vial must be able to withstand temperatures up to 55° C. without deformation while spinning. Suitable materials include glass, polyvinyl chloride, polyethylene, polyamide, polyethylene, polypropylene, polycarbonate, poly(methyl methacrylate (PMMA), polysulfone, polyurethane, and co-polymers of these and other polymers. Specific materials for use with the present disclosure include polypropylene, polycarbonate, and polystyrene. In some embodiments, the rotating growth vial is inexpensively fabricated by, e.g., injection molding or extrusion.

FIGS. 2B-2D show an embodiment of a cell growth module 250 comprising a rotating growth vial 200. FIG. 2B is a perspective view of one embodiment of a cell growth device 250. FIG. 2C depicts a cut-away view of the cell growth device 250 from FIG. 2B. In both figures, the rotating growth vial 200 is seen positioned inside a main housing 226 with the extended lip 202 of the rotating growth vial 200 extending above the main housing 226. Additionally, end housings 222, a lower housing 232, and flanges 224 are indicated in both figures. Flanges 224 are used to attach the cell growth device to heating/cooling means or other structure (not shown). FIG. 2C depicts additional detail. In FIG. 2C, upper bearing 242 and lower bearing 230 are shown positioned in main housing 226. Upper bearing 242 and lower bearing 243 support the vertical load of rotating growth vial 200. Lower housing 232 contains the drive motor 236. The cell growth device of FIG. 2C comprises two light paths: a primary light path 234, and a secondary light path 230. Light path 234 corresponds to light path 210 positioned in the constricted portion of the tapered-to-constricted portion of the rotating growth vial, and light path 230 corresponds to light path 208 in the tapered portion of the tapered-to-constricted portion of the rotating growth vial. Light paths 210 and 208 are not shown in FIG. 2C but may be seen in, e.g., FIG. 2A. In addition to light paths 234 and 230, there is an emission board 228 to illuminate the light path(s), and detector board 246 to detect the light after the light travels through the cell culture liquid in the rotating growth vial.

The motor 236 used to rotate the rotating growth vial 200 in some embodiments is a brushless DC type drive motor with built-in drive controls that can be set to hold a constant revolution per minute (RPM) between 0 and about 3000 RPM. Alternatively, other motor types such as a stepper, servo, brushed DC, and the like can be used. Optionally, the motor 236 may also have direction control to allow reversing of the rotational direction, and a tachometer to sense and report actual RPM. The motor is controlled by a processor (not shown) according to, e.g., standard protocols programmed into the processor and/or user input, and the motor may be configured to vary RPM to cause axial precession of the cell culture thereby enhancing mixing, e.g., to prevent cell aggregation, increase aeration, and optimize cellular respiration.

Main housing 226, end housings 222 and lower housing 232 of the cell growth device 250 may be fabricated from any suitable, robust material including aluminum, stainless steel, and other thermally conductive materials, including plastics. These structures or portions thereof can be created through various techniques, e.g., metal fabrication, injection molding, creation of structural layers that are fused, etc. Whereas the rotating growth vial is envisioned in some embodiments to be reusable but preferably is consumable, the other components of the cell growth device 250 are preferably reusable and can function as a stand-alone benchtop device or, as here, as a module in a multi-module cell processing system.

The processor (not shown) of the cell growth system may be programmed with information to be used as a “blank” or control for the growing cell culture. A “blank” or control is a vessel containing cell growth medium only, which yields 100% transmittance and 0 OD, while the cell sample will deflect light rays and will have a lower percent transmittance and higher OD. As the cells grow in the media and become denser, transmittance will decrease and OD will increase. The processor of the cell growth system may be programmed to use wavelength values for blanks commensurate with the growth media typically used in microbial cell culture. Alternatively, a second spectrophotometer and vessel may be included in the cell growth system, where the second spectrophotometer is used to read a blank at designated intervals.

FIG. 2D illustrates a cell growth device as part of an assembly comprising the cell growth device of FIG. 2B coupled to light source 290, detector 292, and thermal components 294. The rotating growth vial 200 is inserted into the cell growth device. Components of the light source 290 and detector 292 (e.g., such as a photodiode with gain control to cover 5-log) are coupled to the main housing of the cell growth device. The lower housing 232 that houses the motor that rotates the rotating growth vial is illustrated, as is one of the flanges 224 that secures the cell growth device to the assembly. Also illustrated is a Peltier device or thermoelectric cooler 294. In this embodiment, thermal control is accomplished by attachment and electrical integration of the cell growth device 200 to the thermal device 294 via the flange 224 on the base of the lower housing 232. Thermoelectric coolers are capable of “pumping” heat to either side of a junction, either cooling a surface or heating a surface depending on the direction of current flow. In one embodiment, a thermistor is used to measure the temperature of the main housing and then, through a standard electronic proportional-integral-derivative (PID) controller loop, the rotating growth vial 200 is controlled to approximately +/−0.5° C.

In certain embodiments, a rear-mounted power entry module contains the safety fuses and the on-off switch, which when switched on powers the internal AC and DC power supplies (not shown) activating the processor. Measurements of optical densities (OD) at programmed time intervals are accomplished using a 600 nm Light Emitting Diode (LED) (not shown) that has been columnated through an optic into the lower constricted portion of the rotating growth vial which contains the cells of interest. The light continues through a collection optic to the detection system which consists of a (digital) gain-controlled silicone photodiode. Generally, optical density is normally shown as the absolute value of the logarithm with base 10 of the power transmission factors of an optical attenuator: OD=−log 10 (Power out/Power in). Since OD is the measure of optical attenuation—that is, the sum of absorption, scattering, and reflection—the cell growth device OD measurement records the overall power transmission, so as the cells grow and become denser in population the OD (the loss of signal) increases. The OD system is pre-calibrated against OD standards with these values stored in an on-board memory accessible by the measurement program.

In use, cells are inoculated (cells can be pipetted, e.g., from an automated liquid handling system or by a user) into pre-filled growth media of a rotating growth vial by piercing though the foil seal. The programmed software of the cell growth device sets the control temperature for growth, typically 30° C., then slowly starts the rotation of the rotating growth vial. The cell/growth media mixture slowly moves vertically up the wall due to centrifugal force allowing the rotating growth vial to expose a large surface area of the mixture to a normal oxygen environment. The growth monitoring system takes either continuous readings of the OD or OD measurements at pre-set or pre-programmed time intervals. These measurements are stored in internal memory and if requested the software plots the measurements versus time to display a growth curve. If enhanced mixing is required, e.g., to optimize growth conditions, the speed of the vial rotation can be varied to cause an axial precession of the liquid, and/or a complete directional change can be performed at programmed intervals. The growth monitoring can be programmed to automatically terminate the growth stage at a pre-determined OD, and then quickly cool the mixture to a lower temperature to inhibit further growth.

One application for the cell growth device 250 is to constantly measure the optical density of a growing cell culture. One advantage of the described cell growth device is that optical density can be measured continuously (kinetic monitoring) or at specific time intervals; e.g., every 5, 10, 15, 20, 30 45, or 60 seconds, or every 1, 2, 3, 4, 5, 6, 7, 8, 9, or 10 on minutes. While the cell growth device has been described in the context of measuring the optical density (OD) of a growing cell culture, it should, however, be understood by a skilled artisan given the teachings of the present specification that other cell growth parameters can be measured in addition to or instead of cell culture OD. For example, spectroscopy using visible, UV, or near infrared (NIR) light allows monitoring the concentration of nutrients and/or wastes in the cell culture. Additionally, spectroscopic measurements may be used to quantify multiple chemical species simultaneously. Nonsymmetric chemical species may be quantified by identification of characteristic absorbance features in the NIR. Conversely, symmetric chemical species can be readily quantified using Raman spectroscopy. Many critical metabolites, such as glucose, glutamine, ammonia, and lactate have distinct spectral features in the IR, such that they may be easily quantified. The amount and frequencies of light absorbed by the sample can be correlated to the type and concentration of chemical species present in the sample. Each of these measurement types provides specific advantages. FT-NIR provides the greatest light penetration depth and can be used for thicker sample. FT-mid-IR (MIR) provides information that is more easily discernible as being specific for certain analytes as these wavelengths are closer to the fundamental IR absorptions. FT-Raman is advantageous when interference due to water is to be minimized. Other spectral properties can be measured via, e.g., dielectric impedence spectroscopy, visible fluorescence, fluorescence polarization, or luminescence. Additionally, the cell growth device may include additional sensors for measuring, e.g., dissolved oxygen, carbon dioxide, pH, conductivity, and the like.

### The Cell Concentration Module

FIGS. 3A-3I depict variations on one embodiment of a cell concentration/buffer exchange cassette and module that utilizes tangential flow filtration and is configured for use with all cell types, including immortalized cell lines, primary cells and/or stem cells. One embodiment of a cell concentration device described herein operates using tangential flow filtration (TFF), also known as crossflow filtration, in which the majority of the feed flows tangentially over the surface of the filter thereby reducing cake (retentate) formation as compared to dead-end filtration, in which the feed flows into the filter. Secondary flows relative to the main feed are also exploited to generate shear forces that prevent filter cake formation and membrane fouling thus maximizing particle recovery, as described below.

The TFF device described herein was designed to take into account two primary design considerations. First, the geometry of the TFF device leads to filtering the cell culture over a large surface area so as to minimize processing time. Second, the design of the TFF device is configured to minimize filter fouling. FIG. 3A is a general model of tangential flow filtration. The TFF device operates using tangential flow filtration, also known as cross-flow filtration. FIG. 3A shows a system 390 with cells flowing over a membrane 394, where the feed flow of the cells 392 in medium or buffer is parallel to the membrane 394. TFF is different from dead-end filtration where both the feed flow and the pressure drop are perpendicular to a membrane or filter.

FIG. 3B depicts a top view of the lower member of one embodiment of a TFF device/module providing tangential flow filtration. As can be seen in the embodiment of the TFF device of FIG. 3B, TFF device 300 comprises a channel structure 316 comprising a flow channel 302b through which a cell culture is flowed. The channel structure 316 comprises a single flow channel 302b that is horizontally bifurcated by a membrane (not shown) through which buffer or medium may flow, but cells cannot. This particular embodiment comprises an undulating serpentine geometry 314 (i.e., the small “wiggles” in the flow channel 302) and a serpentine “zig-zag” pattern where the flow channel 302 crisscrosses the device from one end at the left of the device to the other end at the right of the device. The serpentine pattern allows for filtration over a high surface area relative to the device size and total channel volume, while the undulating contribution creates a secondary inertial flow to enable effective membrane regeneration preventing membrane fouling. Although an undulating geometry and serpentine pattern are exemplified here, other channel configurations may be used as long as the channel can be bifurcated by a membrane, and as long as the channel configuration provides for flow through the TFF module in alternating directions. In addition to the flow channel 302b, portals 304 and 306 as part of the channel structure 316 can be seen, as well as recesses 308. Portals 304 collect cells passing through the channel on one side of a membrane (not shown) (the “retentate”), and portals 306 collect the medium (“filtrate” or “permeate”) passing through the channel on the opposite side of the membrane (not shown). In this embodiment, recesses 308 accommodate screws or other fasteners (not shown) that allow the components of the TFF device to be secured to one another.

The length 310 and width 312 of the channel structure 316 may vary depending on the volume of the cell culture to be grown and the optical density of the cell culture to be concentrated. The length 310 of the channel structure 316 typically is from 1 mm to 300 mm, or from 50 mm to 250 mm, or from 60 mm to 200 mm, or from 70 mm to 150 mm, or from 80 mm to 100 mm. The width of the channel structure 316 typically is from 1 mm to 120 mm, or from 20 mm to 100 mm, or from 30 mm to 80 mm, or from 40 mm to 70 mm, or from 50 mm to 60 mm. The cross-section configuration of the flow channel 302 may be round, elliptical, oval, square, rectangular, trapezoidal, or irregular. If square, rectangular, or another shape with generally straight sides, the cross section may be from about 10 μm to 1000 μm wide, or from 200 μm to 800 μm wide, or from 300 μm to 700 μm wide, or from 400 μm to 600 μm wide; and from about 10 μm to 1000 μm high, or from 200 μm to 800 μm high, or from 300 μm to 700 μm high, or from 400 μm to 600 μm high. If the cross section of the flow channel 302 is generally round, oval or elliptical, the radius of the channel may be from about 50 μm to 1000 μm in hydraulic radius, or from 5 μm to 800 μm in hydraulic radius, or from 200 μm to 700 μm in hydraulic radius, or from 300 μm to 600 μm wide in hydraulic radius, or from about 200 to 500 μm in hydraulic radius.

When looking at the top view of the TFF device/module of FIG. 3B, note that there are two retentate portals 304 and two filtrate portals 306, where there is one of each type portal at both ends (e.g., the narrow edge) of the device 300. In other embodiments, retentate and filtrate portals can on the same surface of the same member (e.g., upper or lower member), or they can be arranged on the side surfaces of the assembly. Unlike other TFF devices that operate continuously, the TFF device/module described herein uses an alternating method for concentrating cells. The overall workflow for cell concentration using the TFF device/module involves flowing a cell culture or cell sample tangentially through the channel structure. The membrane bifurcating the flow channels retains the cells on one side of the membrane and allows unwanted medium or buffer to flow across the membrane into a filtrate side (e.g., lower member 320) of the device. In this process, a fixed volume of cells in medium or buffer is driven through the device until the cell sample is collected into one of the retentate portals 304, and the medium/buffer that has passed through the membrane is collected through one or both of the filtrate portals 306. All types of prokaryotic and eukaryotic cells—both adherent and non-adherent cells—can be grown in the TFF device. Adherent cells may be grown on beads or other cell scaffolds suspended in medium that flow through the TFF device.

In the cell concentration process, passing the cell sample through the TFF device and collecting the cells in one of the retentate portals 304 while collecting the medium in one of the filtrate portals 306 is considered “one pass” of the cell sample. The transfer between retentate reservoirs “flips” the culture. The retentate and filtrate portals collecting the cells and medium, respectively, for a given pass reside on the same end of TFF device/module 300 with fluidic connections arranged so that there are two distinct flow layers for the retentate and filtrate sides, but if the retentate portal 304 resides on the upper member of device/module 300 (that is, the cells are driven through the channel above the membrane and the filtrate (medium) passes to the portion of the channel below the membrane), the filtrate portal 306 will reside on the lower member of device/module 100 and vice versa (that is, if the cell sample is driven through the channel below the membrane, the filtrate (medium) passes to the portion of the channel above the membrane). This configuration can be seen more clearly in FIGS. 3C-3D, where the retentate flows 360 from the retentate portals 304 and the filtrate flows 370 from the filtrate portals 306.

At the conclusion of a “pass” in the growth concentration process, the cell sample is collected by passing through the retentate portal 304 and into the retentate reservoir (not shown). To initiate another “pass”, the cell sample is passed again through the TFF device, this time in a flow direction that is reversed from the first pass. The cell sample is collected by passing through the retentate portal 304 and into retentate reservoir (not shown) on the opposite end of the device/module from the retentate portal 304 that was used to collect cells during the first pass. Likewise, the medium/buffer that passes through the membrane on the second pass is collected through the filtrate portal 306 on the opposite end of the device/module from the filtrate portal 306 that was used to collect the filtrate during the first pass, or through both portals. This alternating process of passing the retentate (the concentrated cell sample) through the device/module is repeated until the cells have been concentrated to a desired volume, and both filtrate portals can be open during the passes to reduce operating time. In addition, buffer exchange may be effected by adding a desired buffer (or fresh medium) to the cell sample in the retentate reservoir, before initiating another “pass”, and repeating this process until the old medium or buffer is diluted and filtered out and the cells reside in fresh medium or buffer. Note that buffer exchange and cell concentration may (and typically do) take place simultaneously.

FIG. 3C depicts a top view of upper (322) and lower (320) members of an exemplary TFF module. Again, portals 304 and 306 are seen. As noted above, recesses—such as the recesses 308 seen in FIG. 3B—provide a means to secure the components (upper member 322, lower member 320, and membrane 324) of the TFF device/membrane to one another during operation via, e.g., screws or other like fasteners. However, in alterative embodiments an adhesive, such as a pressure sensitive adhesive, or ultrasonic welding, or solvent bonding, may be used to couple the upper member 322, lower member 320, and membrane 324 together. Indeed, one of ordinary skill in the art given the guidance of the present disclosure can find yet other configurations for coupling the components of the TFF device, such as e.g., clamps; mated fittings disposed on the upper and lower members; combination of adhesives, welding, solvent bonding, and mated fittings; and other such fasteners and couplings.

Note that there is one retentate portal and one filtrate portal on each “end” (e.g., the narrow edges) of the TFF device/module. The retentate and filtrate portals on the left side of the device/module will collect cells (flow path at 360) and medium (flow path at 370), respectively, for the same pass. Likewise, the retentate and filtrate portals on the right side of the device/module will collect cells (flow path at 360) and medium (flow path at 370), respectively, for the same pass. In this embodiment, the retentate is collected from portals 304 on the top surface of the TFF device, and filtrate is collected from portals 306 on the bottom surface of the device. The cells are maintained in the TFF flow channel above the membrane 324, while the filtrate (medium) flows through membrane 324 and then through portals 306; thus, the top/retentate portals and bottom/filtrate portals configuration is practical. It should be recognized, however, that other configurations of retentate and filtrate portals may be implemented such as positioning both the retentate and filtrate portals on the side (as opposed to the top and bottom surfaces) of the TFF device. In FIG. 3C, the channel structure 302b can be seen on the bottom member 320 of the TFF device 300. However, in other embodiments, retentate and filtrate portals can reside on the same of the TFF device.

Also seen in FIG. 3C is membrane or filter 324. Filters or membranes appropriate for use in the TFF device/module are those that are solvent resistant, are contamination free during filtration, and are able to retain the types and sizes of cells of interest. For example, pore sizes can be as low as 0.2 μm, however for other cell types, the pore sizes can be as high as 5 μm. Indeed, the pore sizes useful in the TFF device/module include filters with sizes from 0.20 μm, 0.21 μm, 0.22 μm, 0.23 μm, 0.24 μm, 0.25 μm, 0.26 μm, 0.27 μm, 0.28 μm, 0.29 μm, 0.30 μm, 0.31 μm, 0.32 μm, 0.33 μm, 0.34 μm, 0.35 μm, 0.36 μm, 0.37 μm, 0.38 μm, 0.39 μm, 0.40 μm, 0.41 μm, 0.42 μm, 0.43 μm, 0.44 μm, 0.45 μm, 0.46 μm, 0.47 μm, 0.48 μm, 0.49 μm, 0.50 μm and larger. The filters may be fabricated from any suitable non-reactive material including cellulose mixed ester (cellulose nitrate and acetate) (CME), polycarbonate (PC), polyvinylidene fluoride (PVDF), polyethersulfone (PES), polytetrafluoroethylene (PTFE), nylon, glass fiber, or metal substrates as in the case of laser or electrochemical etching. The TFF device shown in FIGS. 3C and 3D do not show a seat in the upper 312 and lower 320 members where the filter 324 can be seated or secured (for example, a seat half the thickness of the filter in each of upper 312 and lower 320 members); however, such a seat is contemplated in some embodiments.

FIG. 3D depicts a bottom view of upper and lower components of the exemplary TFF module shown in FIG. 3C. FIG. 3D depicts a bottom view of upper (322) and lower (320) components of an exemplary TFF module. Again portals 304 and 306 are seen. Note again that there is one retentate portal and one filtrate portal on each end of the device/module. The retentate and filtrate portals on the left side of the device/module will collect cells (flow path at 360) and medium (flow path at 370), respectively, for the same pass. Likewise, the retentate and filtrate portals on the right side of the device/module will collect cells (flow path at 360) and medium (flow path at 370), respectively, for the same pass. In FIG. 3D, the channel structure 302a can be seen on the upper member 322 of the TFF device 300. Thus, looking at FIGS. 3C and 3D, note that there is a channel structure 302 (302a and 302b) in both the upper and lower members, with a membrane 324 between the upper and lower portions of the channel structure. The channel structure 302 of the upper 322 and lower 320 members (302a and 302b, respectively) mate to create the flow channel with the membrane 324 positioned horizontally between the upper and lower members of the flow channel thereby bifurcating the flow channel.

Medium exchange (during cell growth) or buffer exchange (during cell concentration or rendering the cells competent) is performed on the TFF device/module by adding fresh medium to growing cells or a desired buffer to the cells concentrated to a desired volume; for example, after the cells have been concentrated at least 20-fold, 30-fold, 40-fold, 50-fold, 60-fold, 70-fold, 80-fold, 90-fold, 100-fold, 150-fold, 200-fold or more. A desired exchange medium or exchange buffer is added to the cells either by addition to the retentate reservoir or thorough the membrane from the filtrate side and the process of passing the cells through the TFF device 300 is repeated until the cells have been grown to a desired optical density or concentrated to a desired volume in the exchange medium or buffer. This process can be repeated any number of desired times so as to achieve a desired level of exchange of the buffer and a desired volume of cells. The exchange buffer may comprise, e.g., glycerol or sorbitol thereby rendering the cells competent for transformation in addition to decreasing the overall volume of the cell sample.

The TFF device 300 may be fabricated from any robust material in which channels (and channel branches) may be milled including stainless steel, silicon, glass, aluminum, or plastics including cyclic-olefin copolymer (COC), cyclo-olefin polymer (COP), polystyrene, polyvinyl chloride, polyethylene, polyamide, polyethylene, polypropylene, acrylonitrile butadiene, polycarbonate, polyetheretheketone (PEEK), poly(methyl methylacrylate) (PMMA), polysulfone, and polyurethane, and co-polymers of these and other polymers. If the TFF device/module is disposable, preferably it is made of plastic. In some embodiments, the material used to fabricate the TFF device/module is thermally conductive so that the cell culture may be heated or cooled to a desired temperature. In certain embodiments, the TFF device is formed by precision mechanical machining, laser machining, electro discharge machining (for metal devices); wet or dry etching (for silicon devices); dry or wet etching, powder or sandblasting, photostructuring (for glass devices); or thermoforming, injection molding, hot embossing, or laser machining (for plastic devices) using the materials mentioned above that are amenable to this mass production techniques.

FIGS. 3E-3K depict an alternative embodiment of a tangential flow filtration (TFF) device/module. FIG. 3E depicts a configuration of an upper (retentate) member 3022 (on left), a membrane or filter 3024 (middle), and a lower (permeate/filtrate) member 3020 (on the right). In the configuration shown in FIGS. 3E-3, the retentate member 3022 is no longer “upper” and the permeate/filtrate member 3020 is no longer “lower”, as the retentate member 3022 and permeate/filtrate member 3020 are coupled side-to-side as seen in FIGS. 3J and 3K. In FIG. 3E, retentate member 3022 comprises a tangential flow channel 3002, which has a serpentine configuration that initiates at one lower corner of retentate member 3022—specifically at retentate port 3028—traverses across and up then down and across retentate member 3022, ending in the other lower corner of retentate member 3022 at a second retentate port 3028. Also seen on retentate member 3022 is energy director 3091, which circumscribes the region where membrane or filter 3024 is seated. Energy director 3091 in this embodiment mates with and serves to facilitate ultrasonic wending or bonding of retentate member 3022 with permeate/filtrate member 3020 via the energy director component on permeate/filtrate member 3020. Also seen is membrane or filter 3024 has through-holes for retentate ports 3028, which is configured to seat within the circumference of energy directors 3091 between the retentate member 3022 and the permeate/filtrate member 3020. Permeate/filtrate member 3020 comprises, in addition to energy director 3091, through-holes for retentate port 3028 at each bottom corner (which mate with the through-holes for retentate ports 3028 at the bottom corners of membrane 3024 and retentate ports 3028 in retentate member 3022), as well as a tangential flow channel 3002 and a single permeate/filtrate port 3026 positioned at the top and center of permeate/filtrate member 3020. The tangential flow channel 3002 structure in this embodiment has a serpentine configuration and an undulating geometry, although other geometries may be used. In some aspects, the length of the tangential flow channel is from 10 mm to 1000 mm, from 60 mm to 200 mm, or from 80 mm to 100 mm. In some aspects, the width of the channel structure is from 10 mm to 120 mm, from 40 mm to 70 mm, or from 50 mm to 60 mm. In some aspects, the cross section of the tangential flow channel 1202 is rectangular. In some aspects, the cross section of the tangential flow channel 1202 is 5 μm to 1000 μm wide and 5 μm to 1000 μm high, 300 μm to 700 μm wide and 300 μm to 700 μm high, or 400 μm to 600 μm wide and 400 μm to 600 μm high. In other aspects, the cross section of the tangential flow channel 1202 is circular, elliptical, trapezoidal, or oblong, and is 100 μm to 1000 μm in hydraulic radius, 300 μm to 700 μm in hydraulic radius, or 400 μm to 600 μm in hydraulic radius.

FIG. 3F is a side perspective view of a reservoir assembly 3050. The embodiment of FIG. 3F, the retentate member is separate from the reservoir assembly. Reservoir assembly 3050 comprises retentate reservoirs 3052 on either side of a single permeate reservoir 3054. Retentate reservoirs 3052 are used to contain the cells and medium as the cells are transferred through the cell concentration/growth device or module and into the retentate reservoirs during cell concentration and/or growth. Permeate/filtrate reservoir 3054 is used to collect the filtrate fluids removed from the cell culture during cell concentration, or old buffer or medium during cell growth. In the embodiment depicted in FIGS. 3E-3L, buffer or medium is supplied to the permeate/filtrate member from a reagent reservoir separate from the device module. Additionally seen in FIG. 3F are grooves 3032 to accommodate pneumatic ports (not seen), permeate/filtrate port 3026, and retentate port through-holes 3028. The retentate reservoirs are fluidically coupled to the retentate ports 3028, which in turn are fluidically coupled to the portion of the tangential flow channel disposed in the retentate member (not shown). The permeate/filtrate reservoir is fluidically coupled to the permeate/filtrate port 3026 which in turn are fluidically coupled to the portion of the tangential flow channel disposed in permeate/filtrate member (not shown), where the portions of the tangential flow channels are bifurcated by membrane (not shown). In embodiments including the present embodiment, up to 120 mL of cell culture can be grown and/or filtered, or up to 100 mL, 90 mL, 80 mL, 70 mL, 60 mL, 50 mL, 40 mL, 30 mL or 20 mL of cell culture can be grown and/or concentrated.

FIG. 3G depicts a top-down view of the reservoir assembly 3050 shown in FIG. 3F, FIG. 3H depicts a cover 3044 for reservoir assembly 3050 shown in FIGS. 3F, and 3I depicts a gasket 3045 that in operation is disposed on cover 3044 of reservoir assembly 3050 shown in FIG. 3F. FIG. 3G is a top-down view of reservoir assembly 3050, showing two retentate reservoirs 3052, one on either side of permeate reservoir 3054. Also seen are grooves 3032 that will mate with a pneumatic port (not shown), and fluid channels 3034 that reside at the bottom of retentate reservoirs 3052, which fluidically couple the retentate reservoirs 3052 with the retentate ports 3028 (not shown), via the through-holes for the retentate ports in permeate/filtrate member 3024 and membrane 3024 (also not shown). FIG. 3H depicts a cover 3044 that is configured to be disposed upon the top of reservoir assembly 3050. Cover 3044 has round cut-outs at the top of retentate reservoirs 3052 and permeate/filtrate reservoir 3054. Again, at the bottom of retentate reservoirs 3052 fluid channels 3034 can be seen, where fluid channels 3034 fluidically couple retentate reservoirs 3052 with the retentate ports 3028 (not shown). Also shown are three pneumatic ports 3030 for each retentate reservoir 3052 and permeate/filtrate reservoir 3054. FIG. 3I depicts a gasket 3045 that is configured to be disposed upon the cover 3044 of reservoir assembly 3050. Seen are three fluid transfer ports 3042 for each retentate reservoir 3052 and for permeate/filtrate reservoir 3054. Again, three pneumatic ports 3030, for each retentate reservoir 3052 and for permeate/filtrate reservoir 3054, are shown.

FIG. 3J depicts an embodiment of assembled TFF module 3000. Note that in this embodiment of a TFF module the retentate member 3022 is no longer “upper”, and the permeate/filtrate member 3020 is no longer “lower”, as the retentate member 3022 and permeate/filtrate member 3020 are coupled side-to-side with membrane 3024 sandwiched between retentate member 3022 and permeate/filtrate member 3020. Also, retentate member 3022, membrane member 3024, and permeate/filtrate member 3020 are coupled side-to-side with reservoir assembly 3050. Seen are two retentate ports 3028 (which couple the tangential flow channel 3002 in retentate member 3022 to the two retentate reservoirs (not shown), and one permeate/filtrate port 3026, which couples the tangential flow channel 3002 in permeate/filtrate member 3020 to the permeate/filtrate reservoir (not shown). Also seen is tangential flow channel 3002, which is formed by the mating of retentate member 3022 and permeate/filtrate member 3020, with membrane 3024 sandwiched between and bifurcating tangential flow channel 3002. Also seen is energy director 3091, which in this FIG. 3J has been used to ultrasonically weld or couple retentate member 3022 and permeate/filtrate member 3020, surrounding membrane 3024. Cover 3044 can be seen on top of reservoir assembly 3050, and gasket 3045 is disposed upon cover 3044. Gasket 3045 engages with and provides a fluid-tight seal and pneumatic connections with fluid transfer ports 3042 and pneumatic ports 3030, respectively.

FIG. 3K depicts, on the left, an exploded view of the TFF module 3000 shown in FIG. 3J. Seen are components reservoir assembly 3050, a cover 3044 to be disposed on reservoir assembly 3050, a gasket 3045 to be disposed on cover 3044, retentate member 3022, membrane or filter 3024, and permeate/filtrate member 3020. Also seen is permeate/filtrate port 3026, which mates with permeate/filtrate port 3026 on permeate/filtrate reservoir 3054, as well as two retentate ports 3028, which mate with retentate ports 3028 on retentate reservoirs 3052 (where only one retentate reservoir 3052 can be seen clearly in this FIG. 3K). Also seen are through-holes for retentate ports 3028 in membrane 3024 and permeate/filtrate member 3020. FIG. 3K depicts on the left the assembled TFF module 3000 showing length, height, and width dimensions. The assembled TFF device 3000 typically is from 50 to 175 mm in height, or from 75 to 150 mm in height, or from 90 to 120 mm in height; from 50 to 175 mm in length, or from 75 to 150 mm in length, or from 90 to 120 mm in length; and is from 30 to 90 mm in depth, or from 40 to 75 mm in depth, or from about 50 to 60 mm in depth. An exemplary TFF device is 110 mm in height, 120 mm in length, and 55 mm in depth.

Like in other embodiments described herein, the TFF device or module depicted in FIGS. 3E-3K can constantly measure cell culture growth, and in some aspects, cell culture growth is measured via optical density (OD) of the cell culture in one or both of the retentate reservoirs and/or in the flow channel of the TFF device. Optical density may be measured continuously (kinetic monitoring) or at specific time intervals; e.g., every 5, 10, 15, 20, 30 45, or 60 seconds, or every 1, 2, 3, 4, 5, 6, 7, 8, 9, or 10 or so on minutes. Further, the TFF module can adjust growth parameters (temperature, aeration) to have the cells at a desired optical density at a desired time.

FIG. 3L is an exemplary pneumatic block diagram suitable for the TFF module depicted in FIGS. 3E-3K. The pump is connected to two solenoid valves (SV5 and SV6) delivering positive pressure (P) or negative pressure (V). The two solenoid valves SV5 and SV6 couple the pump to the manifold, and two solenoid valves, SV1 and SV2, are connected to the reservoirs (RR1 and RR2). There are also two solenoid valves in reserve (SV3 and SV4). There is a proportional valve (PV2 and PV2), a flow meter (FM1 and FM2), and a pressure sensor (Pressure Sensors 1 and 2) positioned in between each of solenoid valves SV1 and SV2 connecting the pump to the system and the solenoid valves SV1 and SV2 to the reservoirs. The pressure sensors and prop valves work in concert in a feedback loop to maintain the required pressure.

As an alternative to the TFF module described above, a cell concentration module comprising a hollow filter may be employed. Examples of filters suitable for use in the present invention include membrane filters, ceramic filters and metal filters. The filter may be used in any shape; the filter may for example be cylindrical or essentially flat. Preferably, the filter used is a membrane filter, preferably a hollow fiber filter. The term “hollow fiber” is meant a tubular membrane. The internal diameter of the tube is at least 0.1 mm, more preferably at least 0.5 mm, most preferably at least 0.75 mm and preferably the internal diameter of the tube is at most 10 mm, more preferably at most 6 mm, most preferably at most 1 mm. Filter modules comprising hollow fibers are commercially available from various companies, including G.E. Life Sciences (Marlborough, Mass.) and InnovaPrep (Drexel, Mo.). Specific examples of hollow fiber filter systems that can be used, modified or adapted for use in the present methods and systems include, but are not limited to, U.S. Pat. Nos. 9,738,918; 9,593,359; 9,574,977; 9,534,989; 9,446,354; 9,295,824; 8,956,880; 8,758,623; 8,726,744; 8,677,839; 8,677,840; 8,584,536; 8,584,535; and 8,110,112.

### The Cell Transformation Module

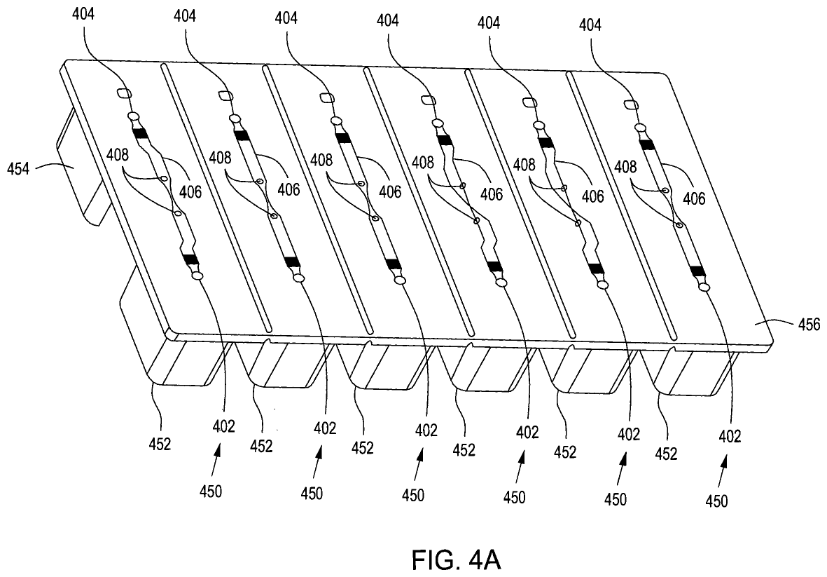

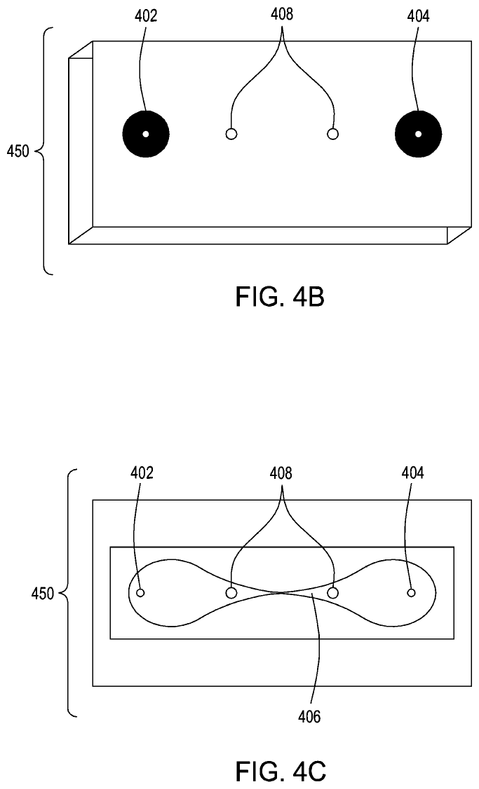

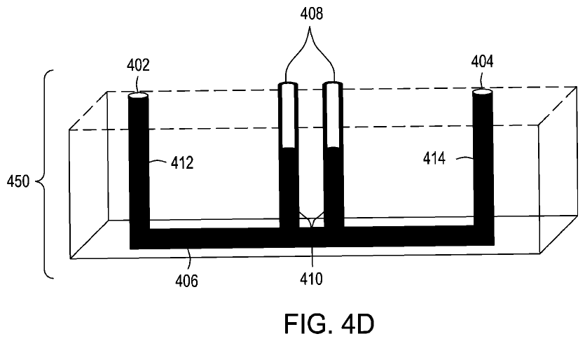

In addition to the modules for cell growth and cell concentration, FIGS. 4A-4E depict variations on one embodiment of a cell transformation module (in this case, a flow through electroporation device) configured to transform microbial cells that may be included in a cell growth/concentration/transformation instrument.

In certain embodiments, some or all of the machinery necessary for editing are introduced using transformation. FIG. 4A is a perspective view of six co-joined flow-through electroporation devices 450. FIG. 4A depicts six flow-through electroporation units 450 arranged on a single substrate 456. Each of the six flow-through electroporation units 450 have wells 452 that define cell sample inlets and wells 454 that define cell sample outlets. Once the six flow-through electroporation units 450 are fabricated, they can be separated from one another (e.g., “snapped apart”) and used one at a time, or alternatively in embodiments two or more flow-through electroporation units 450 can be used in parallel without separation.

The flow-through electroporation devices achieve high efficiency cell electroporation with low toxicity. The flow-through electroporation devices of the disclosure allow for particularly easy integration with robotic liquid handling instrumentation that is typically used in automated systems such as air displacement pipettors. Such automated instrumentation includes, but is not limited to, off-the-shelf automated liquid handling systems from Tecan (Mannedorf, Switzerland), Hamilton (Reno, Nev.), Beckman Coulter (Fort Collins, Colo.), etc.

Generally speaking, microfluidic electroporation—using cell suspension volumes of less than approximately 10 ml and as low as 1 μl—allows more precise control over a transfection or transformation process and permits flexible integration with other cell processing tools compared to bench-scale electroporation devices. Microfluidic electroporation thus provides unique advantages for, e.g., single cell transformation, processing and analysis; multi-unit electroporation device configurations; and integrated, automatic, multi-module cell processing and analysis.

In specific embodiments of the flow-through electroporation devices of the disclosure the toxicity level of the transformation results in greater than 10% viable cells after electroporation, preferably greater than 15%, 20%, 25%, 30%, 35%, 40%, 45%, 50%, 55%, 60%, 70%, 75%, 80%, 85%, 90%, or even 95% viable cells following transformation, depending on the cell type and the nucleic acids being introduced into the cells.

The flow-through electroporation device described in relation to FIGS. 4A-4D comprises a housing with an electroporation chamber, a first electrode and a second electrode configured to engage with an electric pulse generator, by which electrical contacts engage with the electrodes of the electroporation device. In certain embodiments, the electroporation devices are autoclavable and/or disposable, and may be packaged with reagents in a reagent cartridge. The electroporation device may be configured to electroporate cell sample volumes between 1 μl to 2 ml, 10 μl to 1 ml, 25 μl to 750 μl, or 50 μl to 500 μl.

In one exemplary embodiment, FIG. 4B depicts a top view of a flow-through electroporation device 450 having an inlet 402 for introduction of cells and an exogenous reagent to be electroporated into the cells (“cell sample”) and an outlet 404 for the cell sample following electroporation. Electrodes 408 are introduced through electrode channels (not shown) in the device. FIG. 4C shows a cutaway view from the top of flow-through electroporation device 450, with the inlet 402, outlet 404, and electrodes 408 positioned with respect to a constriction in flow channel 406. A side cutaway view of the bottom portion of flow-through electroporation device 450 in FIG. 4D illustrates that electrodes 408 in this embodiment are positioned in electrode channels 410 and perpendicular to flow channel 406 such that the cell sample flows from the inlet channel 412 through the flow channel 406 to the outlet channel 414, and in the process the cell sample flows into the electrode channels 410 to be in contact with electrodes 408. In this aspect, the inlet channel, outlet channel and electrode channels all originate from the top planar side of the device; however, the flow-through electroporation architecture depicted in FIGS. 4B-4D is but one architecture useful with the reagent cartridges described herein. Additional electrode architectures are described, e.g., in U.S. Ser. No. 16/147,120, filed 24 Sep. 2018; Ser. No. 16/147,865, filed 30 Sep. 2018; and Ser. No. 16/147,871, filed 30 Sep. 2018.

### The Reagent Cartridge

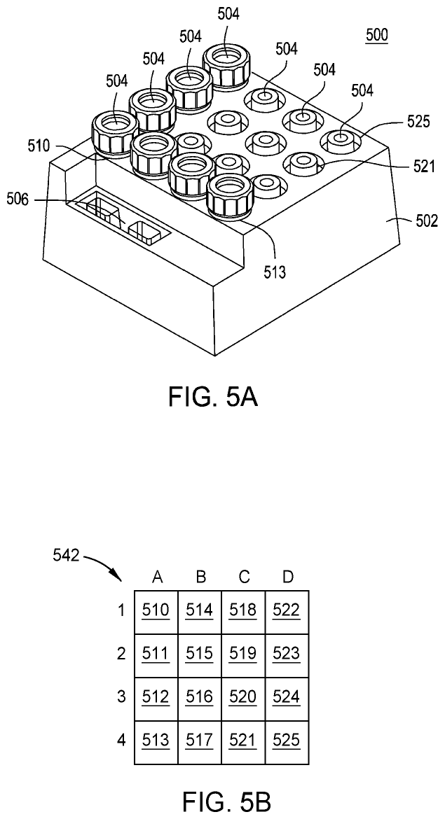

FIG. 5A depicts an exemplary combination reagent cartridge and electroporation device 500 (“cartridge”) that may be used in an automated multi-module cell processing instrument. Cartridge 500 comprises a body 502, and reagent receptacles or reservoirs 504. Additionally, cartridge 500 comprises an electroporation device 506 (an exemplary embodiment of which is described in detail in relation to FIGS. 4A-4E), which is preferably a flow-through electroporation device. Cartridge 500 may be disposable, or may be configured to be reused. Preferably, cartridge 500 is disposable. Cartridge 500 may be made from any suitable material, including stainless steel, aluminum, or plastics including polyvinyl chloride, cyclic olefin copolymer (COC), polyethylene, polyamide, polyethylene, polypropylene, acrylonitrile butadiene, polycarbonate, polyetheretheketone (PEEK), poly(methyl methylacrylate) (PMMA), polysulfone, and polyurethane, and co-polymers of these and other polymers. If the cartridge is disposable, preferably it is made of plastic. Preferably the material used to fabricate the cartridge is thermally conductive, as in certain embodiments the cartridge 500 contacts a thermal device (not shown) that heats or cools reagents in the reagent receptacles or reservoirs 504. In some embodiments, the thermal device is a Peltier device or thermoelectric cooler. Reagent receptacles or reservoirs 504 may be receptacles into which individual tubes of reagents are inserted as shown in FIG. 5A, receptacles into which one or more multiple co-joined tubes are inserted, or the reagent receptacles may hold the reagents without inserted tubes with the reagents dispensed directly into the receptacles or reservoirs. Additionally, the receptacles in a reagent cartridge may be configured for any combination of tubes, co-joined tubes, and direct-fill of reagents.

In one embodiment, the reagent receptacles or reservoirs 504 of reagent cartridge 500 are configured to hold various size tubes, including, e.g., 250 ml tubes, 25 ml tubes, 10 ml tubes, 5 ml tubes, and Eppendorf or microcentrifuge tubes. In yet another embodiment, all receptacles may be configured to hold the same size tube, e.g., 5 ml tubes, and reservoir inserts may be used to accommodate smaller tubes in the reagent reservoir. In yet another embodiment—particularly in an embodiment where the reagent cartridge is disposable—the reagent reservoirs hold reagents without inserted tubes. In this disposable embodiment, the reagent cartridge may be part of a kit, where the reagent cartridge is pre-filled with reagents and the receptacles or reservoirs sealed with, e.g., foil, heat seal acrylic or the like and presented to a consumer where the reagent cartridge can then be used in an automated multi-module cell processing instrument. The reagents contained in the reagent cartridge will vary depending on workflow; that is, the reagents will vary depending on the processes to which the cells are subjected in the automated multi-module cell processing instrument.

FIG. 5B depicts an exemplary matrix configuration 540 for the reagents contained in the reagent cartridges of FIG. 5A; where this matrix embodiment is a 4×4 reagent matrix. Through a matrix configuration, a user (or programmed processor) can locate the proper reagent for a given process. That is, reagents such as cell samples, enzymes, buffers, nucleic acid vectors, expression cassettes, reaction components (such as, e.g., MgCl₂, dNTPs, isothermal nucleic acid assembly reagents, Gap Repair reagents, and the like), wash solutions, ethanol, and magnetic beads for nucleic acid purification and isolation, etc. are positioned in the matrix 540 at a known position. For example, reagents are located at positions A1 (510), A2 (511), A3 (512), A4 (513), B1 (514), B2 (515) and so on through, in this embodiment, position D4 (525). FIG. 5A is labeled to show where several reservoirs 504 correspond to matrix 540: See receptacles 510, 513, 521 and 525. Although the reagent cartridge 500 of FIG. 5A and the matrix configuration 540 of FIG. 5B shows a 4×4 matrix, matrices of the reagent cartridge and electroporation devices can be any configuration, such as, e.g., 2×2, 2×3, 2×4, 2×5, 2×6, 3×3, 3×5, 4×6, 6×7, or any other configuration, including asymmetric configurations, or two or more different matrices depending on the reagents needed for the intended workflow. Note in FIG. 4A the matrix configuration is a 5×3+1 matrix.

In preferred embodiments of reagent cartridge 500 shown in FIG. 5A, the reagent cartridge comprises a script (not shown) readable by a processor (not shown) for dispensing the reagents via a liquid handling device (not shown) and controlling the electroporation device contained within reagent cartridge 500. Also, the reagent cartridge 500 as one component in an automated multi-module cell processing instrument may comprise a script specifying two, three, four, five, ten or more processes performed by the automated multi-module cell processing instrument, or even specify all processes performed by the automated multi-module cell processing instrument. In certain embodiments, the reagent cartridge is disposable and is pre-packaged with reagents tailored to performing specific cell processing protocols, e.g., genome editing or protein production. Because the reagent cartridge contents vary while components of the automated multi-module cell processing instrument may not, the script associated with a particular reagent cartridge matches the reagents used and cell processes performed. Thus, e.g., reagent cartridges may be pre-packaged with reagents for genome editing and a script that specifies the process steps for performing genome editing in an automated multi-module cell processing instrument such as described in relation to FIGS. 1A-1D. For example, the reagent cartridge may comprise a script to pipette electrocompetent cells from reservoir A2 (511), transfer the cells to the electroporation device 506, pipette a nucleic acid solution comprising an editing vector from reservoir C3 (520), transfer the nucleic acid solution to the electroporation device, initiate the electroporation process for a specified time, then move the porated cells to a reservoir D4 (525) in the reagent cassette or to another module such as the rotating growth vial (118 or 120 of FIG. 1A) in the automated multi-module cell processing instrument in FIG. 1A. In another example, the reagent cartridge may comprise a script to pipette transfer of a nucleic acid solution comprising a vector from reservoir C3 (520), nucleic acid solution comprising editing oligonucleotide cassettes in reservoir C4 (521), and isothermal nucleic acid assembly reaction mix from A1 (510) to the isothermal nucleic acid assembly/desalting reservoir (414 of FIG. 4A). The script may also specify process steps performed by other modules in the automated multi-module cell processing instrument. For example, the script may specify that the isothermal nucleic acid assembly/desalting module be heated to 50° C. for 30 min to generate an assembled isothermal nucleic acid product; and desalting of the assembled isothermal nucleic acid product via magnetic bead-based nucleic acid purification involving a series of pipette transfers and mixing of magnetic beads in reservoir B2 (515), ethanol wash in reservoir B3 (516), and water in reservoir C1 (518) to the isothermal nucleic acid assembly/desalting reservoir (114 of FIG. 1A).

### Use of the Automated Multi-Module Microbial Cell Processing Instrument

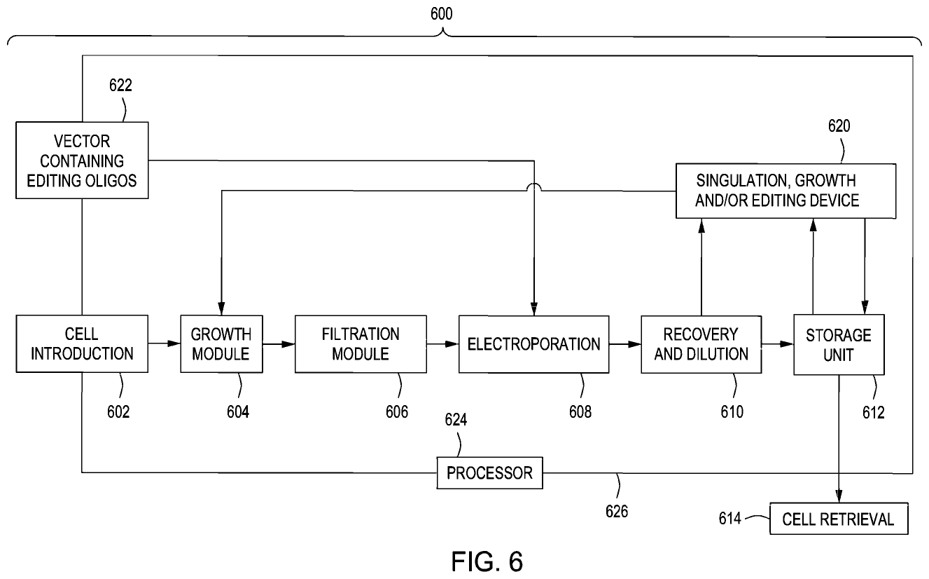

FIG. 6 illustrates an embodiment of a multi-module cell processing instrument. This embodiment depicts an exemplary system that performs recursive gene editing on a microbial cell population, e.g., bacterial cells. The cell processing instrument 600 may include a housing 626, a reservoir 612 for storing cells to be transformed or transfected, and a cell growth module (comprising, e.g., a rotating growth vial) 604. The cells to be transformed are transferred from a reservoir to the cell growth module to be cultured until the cells hit a target OD. Once the cells hit the target OD, the growth module may cool or freeze the cells for later processing or transfer the cells to a cell concentration module 606 where the cells are subjected to buffer exchange and rendered electrocompetent, and the volume of the cells may be reduced substantially. Once the cells have been concentrated to an appropriate volume, the cells are transferred to electroporation device 608. In addition to the reservoir for storing cells 612, the multi-module cell processing instrument includes a reservoir for storing the vector pre-assembled with editing oligonucleotide cassettes 622. The pre-assembled nucleic acid vectors are transferred to the electroporation device 608, which already contains the cell culture grown to a target OD. In the electroporation device 608, the nucleic acids are electroporated into the cells. Following electroporation, the cells are transferred into an optional recovery module 610, where the cells recover briefly post-transformation.

After recovery, the cells may be transferred to a storage module 612, where the cells can be stored at, e.g., 4° C. for later processing, or the cells may be diluted and transferred to an incubation and growth module 620. In some aspects, the cells are transferred from the storage module to a retrieval reservoir 614.

In the incubation and growth module 620, the cells are arrayed such that there is an average of one cell per microwell. The arrayed cells may be in selection medium to select for cells that have been transformed or transfected with the editing vector(s). Once singulated, the cells grow through 2-50 doublings and establish colonies. Editing is then initiated and allowed to proceed, the cells are allowed to grow to terminal size (e.g., normalization of the colonies) in the microwells and then are treated to conditions that cure the editing vector from this round. Once cured, the cells can be flushed out of the microwells and pooled, then transferred to the storage (or recovery) unit 612 or can be transferred back to the growth module 604 for another round of editing. In between pooling and transfer to a growth module, there typically is one or more additional steps, such as cell recovery, medium exchange (rendering the cells electrocompetent), cell concentration (typically concurrently with medium exchange by, e.g., filtration. Note the selection/singulation/growth/incubation/editing/normalization and curing modules may be the same module, where all processes are performed in, e.g., a solid wall device, or selection and/or dilution may take place in a separate vessel before the cells are transferred to the growth/editing module. In some aspects, the cells are singulated or partitioned in smaller cell groups (e.g., 2-600 cells) for growth and/or editing, as described in, e.g., U.S. Pat. Nos. 10,253,316 and 10,532,324.

In other aspects, the cells may be pooled after normalization, transferred to a separate vessel, and cured in the separate vessel. As an alternative to singulation in, e.g., a solid wall device, the transformed cells may be grown in—and editing can proceed in—bulk liquid as described above in U.S. Ser. No. 68/795,739, filed 23 Jan. 2019. Once the putatively-edited cells are pooled, they may be subjected to another round of editing, beginning with growth, cell concentration and treatment to render electrocompetent, and transformation by yet another donor nucleic acid in another editing cassette via the electroporation module 608.

In electroporation device 608, the microbial cells selected from the first round of editing are transformed by a second set of editing oligos (or other type of oligos) and the cycle is repeated until the cells have been transformed and edited by a desired number of, e.g., editing cassettes or CF editing cassettes. The multi-module cell processing instrument exemplified in FIG. 6 is controlled by a processor 624 configured to operate the instrument based on user input or is controlled by one or more scripts including at least one script associated with the reagent cartridge. The processor 624 may control the timing, duration, and temperature of various processes, the dispensing of reagents, and other operations of the various modules of the instrument 600. For example, a script or the processor may control the dispensing of cells, reagents, vectors, and editing oligonucleotides; which editing oligonucleotides are used for cell editing and in what order; the time, temperature and other conditions used in the recovery and expression module, the wavelength at which OD is read in the cell growth module, the target OD to which the cells are grown, and the target time at which the cells will reach the target OD. In addition, the processor may be programmed to notify a user (e.g., via an application) as to the progress of the cells in the automated multi-module cell processing instrument.

It should be apparent to one of ordinary skill in the art given the present disclosure that the process described may be recursive and multiplexed; that is, cells may go through the workflow described in relation to FIG. 6, then the resulting edited culture may go through another (or several or many) rounds of additional editing (e.g., recursive editing) with different editing vectors. For example, the cells from round 1 of editing may be diluted and an aliquot of the edited cells edited by editing vector A may be combined with editing vector B, an aliquot of the edited cells edited by editing vector A may be combined with editing vector C, an aliquot of the edited cells edited by editing vector A may be combined with editing vector D, and so on for a second round of editing. After round two, an aliquot of each of the double-edited cells may be subjected to a third round of editing, where, e.g., aliquots of each of the AB-, AC-, AD-edited cells are combined with additional editing vectors, such as editing vectors X, Y, and Z. That is that double-edited cells AB may be combined with and edited by vectors X, Y, and Z to produce triple-edited edited cells ABX, ABY, and ABZ; double-edited cells AC may be combined with and edited by vectors X, Y, and Z to produce triple-edited cells ACX, ACY, and ACZ; and double-edited cells AD may be combined with and edited by vectors X, Y, and Z to produce triple-edited cells ADX, ADY, and ADZ, and so on. In this process, many permutations and combinations of edits can be executed, leading to very diverse cell populations and cell libraries. In any recursive process, it is advantageous to “cure” the previous engine and editing vectors (or single engine+editing vector in a single vector system). “Curing” is a process in which one or more vectors used in the prior round of editing is eliminated from the transformed cells.

Curing can be accomplished by, e.g., cleaving the vector(s) using a curing plasmid thereby rendering the editing and/or engine vector (or single, combined engine/editing vector) nonfunctional; diluting the vector(s) in the cell population via cell growth (that is, the more growth cycles the cells go through, the fewer daughter cells will retain the editing or engine vector(s)), or by, e.g., utilizing a heat-sensitive origin of replication on the editing or engine vector (or combined engine+editing vector). The conditions for curing will depend on the mechanism used for curing; that is, in this example, how the curing plasmid cleaves the editing and/or engine vector.

### Vector Design Strategy

The exemplary vector designs below may be utilized according the methods described herein to edit of cells for bioproduction of antibiotics that overcome antibiotic resistance. In some aspects, the vector designs below may be utilized to engineer cells simultaneously encoding a first protein that facilitates production of an antibiotic and a second protein that facilities resistance to the produced antibiotic. As a result, the edited cells edited may serve as rapid screening to produce and screen for novel antibiotics that escape known and novel resistance pathways.

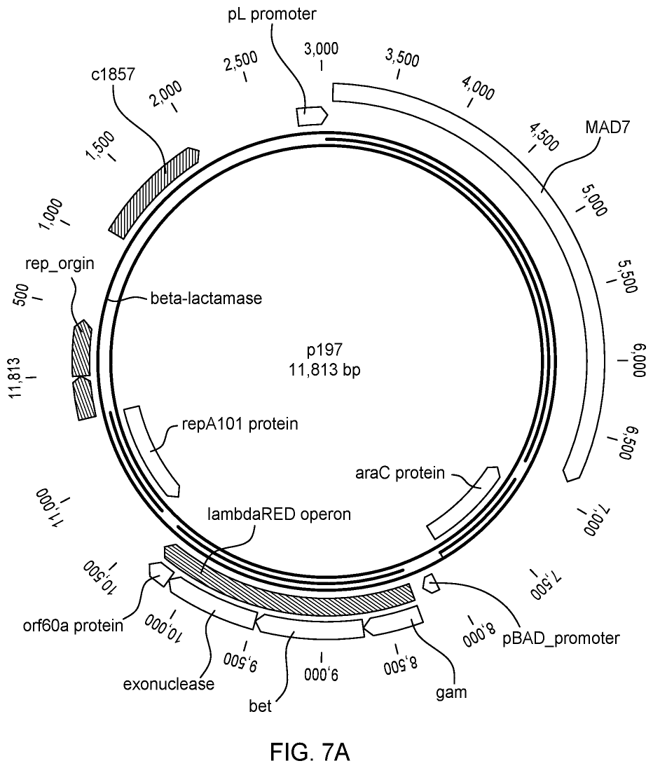

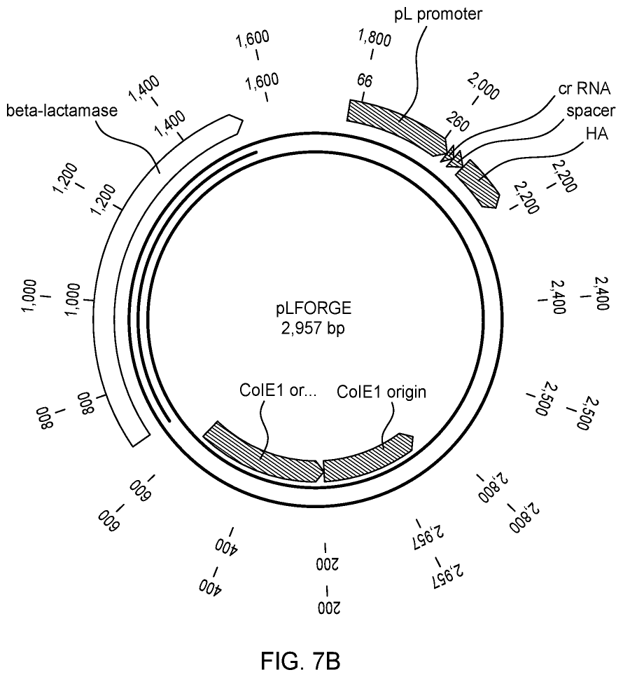

Genetic variants of the present disclosure may be formed by nucleic acid-guided nuclease editing of up to hundreds of thousands of loci in desired strains of bacteria harboring an engine plasmid such as that shown in FIG. 7A (an exemplary transformed E. coli MG1655 strain is depicted and is hereafter referred to as E. coli strain EC83). The engine plasmid comprises a coding sequence for, e.g., a nuclease (MAD7) or CF enzyme under the control of an inducible promoter (pL promoter), a λ Red operon recombineering system under the control of an inducible promoter (pBAD—inducible by the addition of arabinose in a cell growth medium), a c1857 gene under the control of a constitutive promoter, as well as an optional selection marker and an origin of replication. The λ Red recombineering system repairs double-stranded breaks resulting from a cut by the MAD7 nuclease. The c1857 gene, at 30° C., actively represses the pL promoter (which drives the expression of the MAD7 nuclease); however, at 42° C., the c1857 repressor gene unfolds or degrades, and in this state the c1857 repressor protein can no longer repress the pL promoter leading to active transcription of the coding sequence for the MAD7 nuclease.

FIG. 7B depicts an exemplary editing plasmid comprising an editing cassette (crRNA, spacer, desired edit, and homology arm(s) “HA”) or CF editing cassette driven by an inducible promoter (pL), a selection marker, and an origin of replication. Note that FIGS. 7A and 7B are only exemplary. Further details regarding FIGS. 7A and 7B may be found in U.S. patent application Ser. No. 16/904,827, which is incorporated herein by reference for all purposes.

### Antibiotic Synthesis and Screening

The present disclosure is directed to the editing of organisms for enhanced biosynthesis and rapid screening of novel antibiotic compounds. Currently, antibiotic development and screening is an onerous, multistep process that requires a great deal of time and resources. The present disclosure provides devices, instruments, and methods, including automated methods, for enhanced production and efficient screening of such biosynthesized antibiotics. More particularly, the present disclosure provides for accelerated biosynthesis and screening at a single-cell scale.

Described below are methods for achieving such accelerated biosynthesis and screening at a single-cell scale, which generally comprise three iterative phases: (1) initial or base strain development; (2) pathway and gene editing for modifying biosynthesis of antibiotics; and (3) selection accounting for identifying variants that create antibiotics that escape known resistance pathways.

### Initial Antibiotic Synthesis

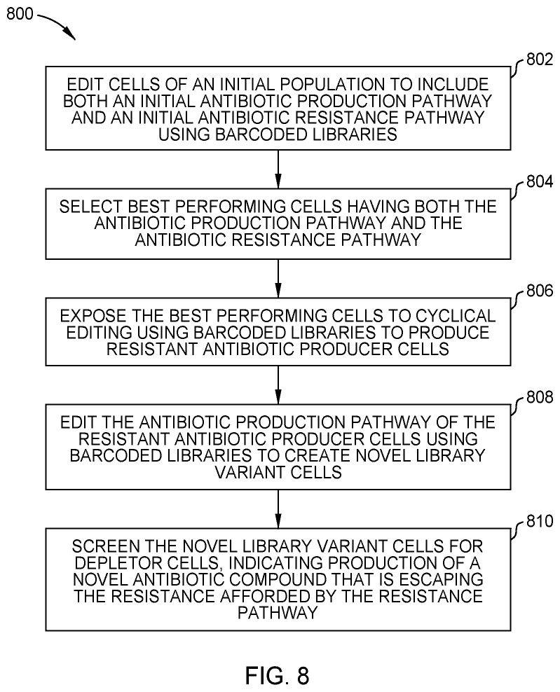

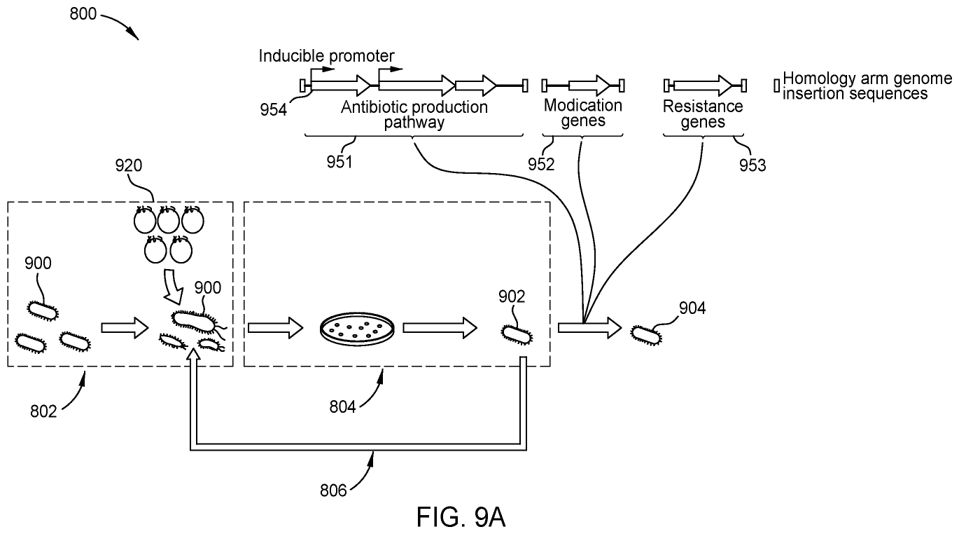

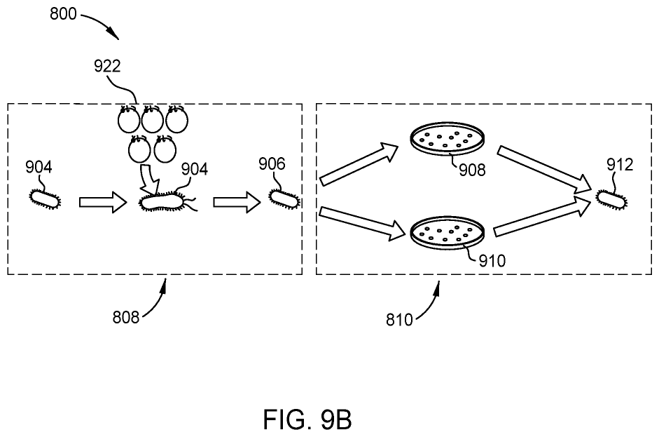

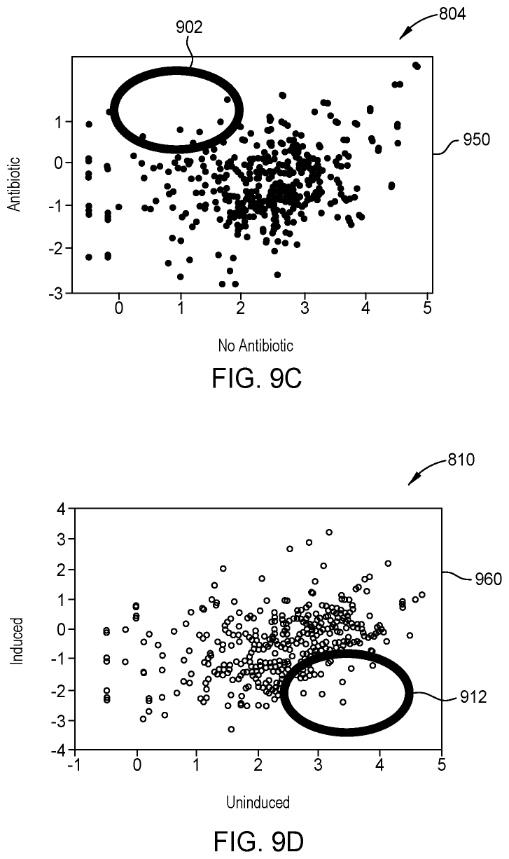

FIG. 8 depicts a flow diagram of a method 800 for initial biosynthesis of novel and potent antibiotic compound candidates, according to certain embodiments. FIGS. 9A-9D schematically and graphically depict various stages of the method 800 represented in FIG. 8. Accordingly, FIG. 8 and FIGS. 9A-9D are herein described together for clarity.

The method 800 begins at operation 802 and FIG. 9A, wherein an initial bacterial population 900 is genetically edited using designed mutagenesis libraries 920 and the automated methods described herein, e.g., multiplexed nuclease-directed editing, such that its genome and/or episome encode both a production pathway for a desired antibiotic platform as well as resistance pathway enabling resistance to the desired antibiotic. Conventionally, antibiotic production pathways and resistance pathways have been transferred between organisms separately, such that the modified organism(s) encoded either antibiotic production genes or antibiotic resistance genes. Here, both antibiotic production and resistance pathways, either of which may be heterologous to the bacterial population 900, are combined into a single strain to enable subsequent screening at the single-cell scale, as compared to the otherwise onerous microtiter plate scale. Examples of suitable bacterial strains for the initial bacterial population 900 include E. coli K-2 MG1655, as well as other strains of the same or other species. Examples of suitable antibiotics for use herein include penicillin, vancomycin, ampicillin, streptomycin, erythromycin, chloramphenicol, kanamycin, tetracycline, gentamicin, bleomycin, puromycin, hygromycin, blasticidin, and the like.

In certain embodiments, cells of the bacterial population 900 are edited to include one or more antibiotic production genes, such as two, three, or four or more antibiotic production genes, e.g., eight or more antibiotic production genes, that encode for proteins (e.g., enzymes) to synthesize compounds that would have antibiotic effects on the bacterial cells 900 if not for the co-encoded resistance pathway. The bacterial population 900 may be engineered such that the antibiotic production genes are either constitutively expressed, or one or more production genes are expressed by induction with a small molecule (such as IPTG, via an inducible promoter). In certain embodiments, the encoded proteins may be part of one or more antibiotic production pathways that synthesize one or more antibiotic compounds, such as two, three, or four or more antibiotic compounds. For example, the bacterial population 900 may be edited to encode for proteins in two or more antibiotic production pathways that generate one or more antibiotic compounds. Each encoded antibiotic production protein is generally encoded as a single copy per cell, either as inserted into the genomes of bacterial population 900, or as replicated on single-copy plasmids, e.g., bacterial artificial chromosomes (BAC).

Similarly, cells of the bacterial population 900 are edited to include one or more antibiotic resistance genes, such as two, three, or four or more antibiotic resistance genes, e.g., eight or more antibiotic resistance genes, that encode for proteins that facilitate resistance of the bacterial population 900 to the antibiotic compounds produced by the antibiotic production pathway. The bacterial population 900 may be engineered such that the antibiotic resistance genes are either constitutively expressed, or expressed by induction with a small molecule, e.g., IPTG. In certain embodiments, the encoded proteins may be part of one or more antibiotic resistance pathways that provide resistance to one or more of the produced antibiotic compounds, such as two or more antibiotic resistance pathways that provide resistance to one or more of the produced antibiotic compounds. In certain embodiments, the encoded resistance pathways are multidrug resistance pathways. Like the antibiotic production proteins, each encoded resistance protein is generally encoded as a single copy per cell, either as inserted into the genomes of bacterial population 900, or as replicated on single-copy plasmids, e.g., a BAC.

In addition to providing the bacterial population 900 with copies of antibiotic production and resistance pathways, the editing at operation 802 may further introduce targeted and genome-wide mutagenesis across one or more additional genes and/or pathways through single edits and/or combinatorial edits at up to hundreds of thousands of loci. In certain embodiments, the designed mutagenesis libraries 920 utilized for operation 802 are designed to introduce up to gene-scale inserts that increase expression of one or more genes and/or pathways, decrease expression of one or more genes and/or pathways, and/or introduce evolved and/or known mutations into cells of the bacterial population 900 to facilitate strain differences between edited cells thereof. The designed mutagenesis libraries 920 further comprise one or more nucleic acid barcodes which may be cloned into the cells of the bacterial population 900, thus facilitating tracking of edits in each cell by the presence or absence of the barcodes; alternatively, the editing (and/or engine) plasmids may carry the barcode, therefore not requiring introduction of the nucleic acid barcodes into the cell genomes. Often, the barcode is included in the editing cassette (e.g., CF editing cassette). Each barcode may identify a one-to-one relationship with one or more intended edits that can be determined through sequencing of the bacterial population 900 before and after each editing process, as well as before and/or after each operation of the current method.

At operation 804, the best performing cells 902 of the bacterial population 900, e.g., having both antibiotic production genes and antibiotic resistance genes conferring resistance to the produced antibiotic compounds, are selected and verified. Introduction of the antibiotic production genes may be separately verified by, e.g., induction of the antibiotic production proteins to show toxicity of the one or more produced antibiotic compounds. Conversely, introduction of the antibiotic resistance genes may be separately verified by, e.g., incubating the bacterial population 900 in the presence of the one or more antibiotic compounds above the minimum inhibitory concentration (MIC) of the initial bacterial population 900 without the resistance gene(s). Introduction of both antibiotic production and resistance pathways may be verified by analyzing growth rates of the bacterial population 900 with and without antibiotic production expression, as depicted by graph 950 in FIG. 9C, and may further be verified by whole genome sequencing, mass spectrometry protein analysis, and/or metabolite analysis.

Upon selection of the best performing cells 902, the antibiotic production and resistance edits are transferred into the genome of the bacterial population 900 by cyclic editing and selection of the cells 902 at operation 806, which may further introduce additional targeted and genome-wide mutagenesis across one or more additional genes and/or pathways through single and/or combinatorial edits with designed mutagenesis libraries 920. As a result, resistant antibiotic producer cells 904 are synthesized each having one or more antibiotic production pathways 951 to produce the desired antibiotic compound(s) (which may be under the control of an inducible promoter 954), one or more modification genes 952 (e.g., methyltransferases, carboxyltransferases, and the like), as well as one or more resistance pathways 953 to provide resistance to the produced antibiotic compound.

At operation 808 shown in FIGS. 8 and 9B, the resistant antibiotic producer cells 904 are subjected to further editing with designed mutagenesis libraries 922 to create novel library variant cells 906 producing novel antibiotic compounds upon induction of the antibiotic production pathways 951. Accordingly, the designed mutagenesis libraries 922 include targeted mutations to the antibiotic production pathways 951 previously introduced into the resistance antibiotic producer cells 904 to diversify antibiotic compounds produced thereby, as well as targeted mutations to the modification genes 952 and nucleic acid barcodes to enable quantitative tracking of edits. Similar to operation 802, the precise and targeted nature of the editing at operation 808 enables multiplexed and genome-wide mutagenesis across one or more genes and/or pathways through single edits and/or combinatorial edits, thus enabling the antibiotic production pathway be modified for maximum diversity of antibiotic compound production by the resistant antibiotic producer cells 904. In certain embodiments, the designed mutagenesis libraries 922 are designed for active site mutagenesis of antibiotic production enzymes encoded by the antibiotic production pathways 951, active site mutagenesis of modification enzymes encoded by the modification genes 952, as well as saturation mutagenesis. As a result of operation 808, modifications to the antibiotic production pathways 951 may be interrogated, while the resistance pathways 953 are maintained.

At operation 810, the novel library variant cells 906 are split into replicates of induced samples 908 and uninduced samples 910 and are selected for reproducible cell depletion (i.e., cell death) as shown in graph 960 of FIG. 9D, which indicates that a novel antibiotic compound is being produced and escaping the resistance afforded by the encoded resistance pathways 953. Depletor cells 912, or cells being reproducibly depleted upon induction, are then utilized for further evaluation and antibiotic compound identification and isolation.

Generally, sequencing of nucleic acids, such as the nucleic acid barcodes housed in the editing cassettes (e.g., CF editing cassettes), is carried out before and/or after each of the operations of method 800 to facilitate tracking of edits to the initial bacterial population. For example, before and after editing at operations 802 and 808, the bacterial cells 900 and 904, respectively, as well as the designed mutagenesis libraries 920 and 922, are sequenced to ensure input library representation is accounted for before and after transformation of the designed libraries into the cells. After editing, the cells are collected and thereafter sequenced again. Sequencing during the method 800 may generally include any suitable type of nucleic acid sequencing process, such as barcode amplicon sequencing, or pooled whole genome sequencing. The sequencing may be carried out utilizing unique molecular identifiers in primers between sequencing adapters that facilitate quantitative discovery of the nucleic acid barcodes and edits within the cell population.

In certain embodiments, the novel library variant cells 906 are sequenced at one or more time points following transformation at operation 808. For example, in certain embodiments, the novel library variant cells 906 are first sequenced immediately after transformation, and then again upon growth for a desired amount of time, such as, e.g., after one generation of growth, two generations of growth, or more than two generations of growth. In certain embodiments, the novel library variant cells 906 are further sequenced at a third time point after induction of the antibiotic production pathways 951 at operation 810, such as, e.g., after one generation of growth after induction, two generations of growth after induction, or more than two generations of growth after induction. Nucleic acid barcodes present in a sequenced population after outgrowth, or after outgrowth and pathway induction, are compared against an initial nucleic acid barcode population. Nucleic acid barcodes that are under-represented in the final cell population (e.g., after final sequencing) indicate novel antibiotic production pathways yielding novel antibiotic compounds that overcome the resistance pathways 953.

### Further Antibiotic Diversification

As previously described, the depletor cells 912 identified through method 800 indicate that a novel antibiotic compound is being produced and escaping the resistance afforded by the encoded resistance pathways 953. After selection and verification of novel antibiotic compounds and pathways from depletor cells 912, the resistance pathways 953 may also be edited in a combinatorial fashion to increase antibiotic resistance of the cells and facilitate further diversification of antibiotic production pathways 951 away from the depletor cells 912.

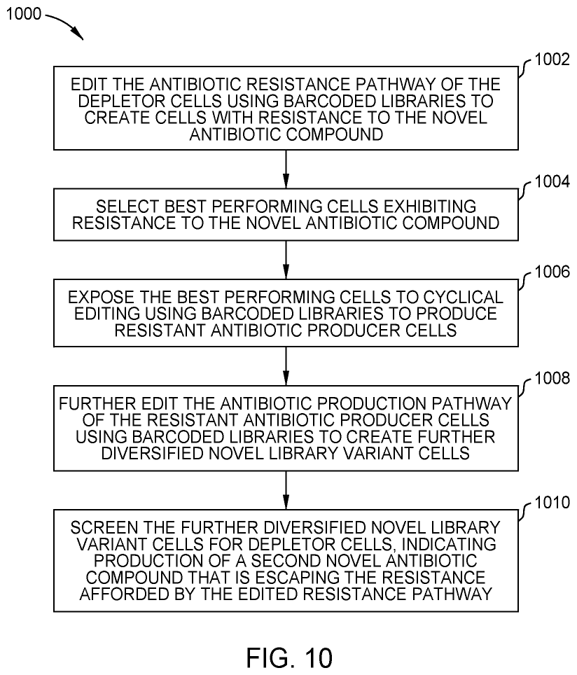

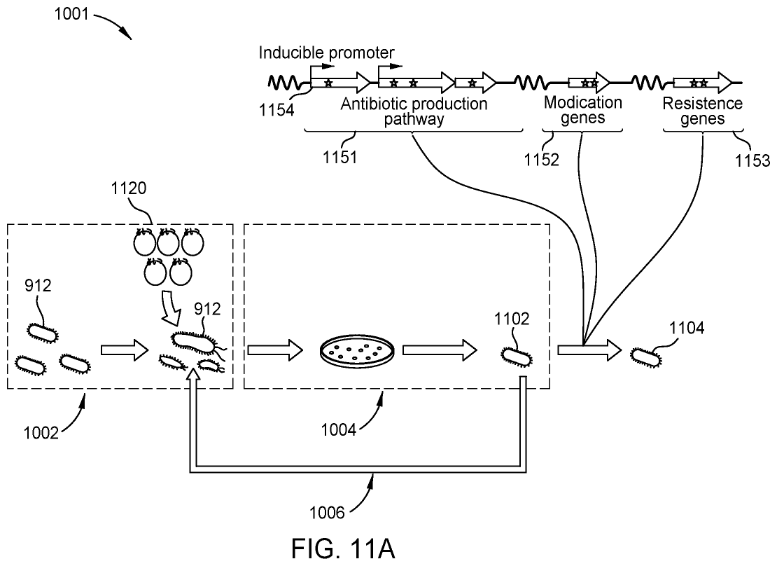

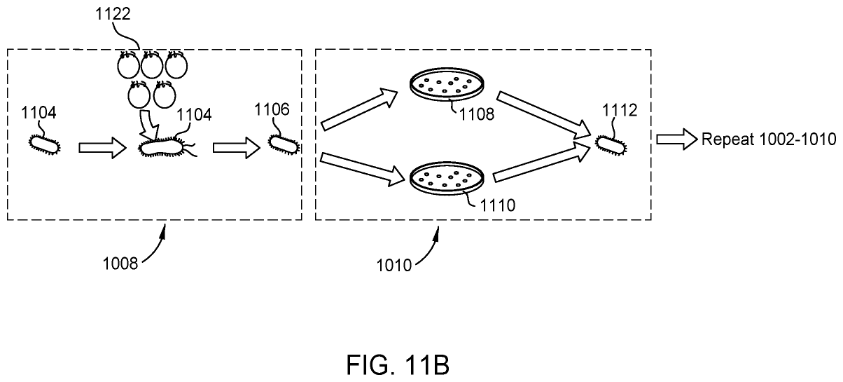

FIG. 10 depicts a flow diagram of a method 1000 for modifying the resistance pathways 953 of bacterial cells selected from the method 800 above, e.g., depletor cells 912, and biosynthesis of novel and diversified antibiotic compounds thereby. FIGS. 11A and 11B schematically depict various stages of the method 1000 represented in FIG. 10. Accordingly, FIG. 10 and FIGS. 11A-11B are herein described together for clarity.

The method 1000 begins at operation 1002 and FIG. 11A, wherein depletor cells 912 are genetically edited to increase resistance using designed mutagenesis libraries 1120 and automated methods described herein, e.g., multiplexed nuclease-directed editing via the CREATE platform. The designed mutagenesis libraries 1120 include targeted mutations to the resistance pathways 953 previously introduced into the cells during method 800 to diversify the resistance pathways 953 and facilitate resistance to the novel antibiotic compounds produced by the depletor cells 912. The designed mutagenesis libraries 1120 further include nucleic acid barcodes to enable quantitative tracking of edits, and in certain embodiments, targeted mutations to the modification genes 952. The libraries 1120 may be designed to introduce targeted and genome-wide mutagenesis across one or more additional genes and/or pathways through single edits and/or combinatorial edits at up to hundreds of thousands of loci. In certain embodiments, the designed mutagenesis libraries 1120 utilized for operation 1002 are designed to introduce up to gene-scale inserts to increase expression of one or more genes and/or pathways, decrease expression of one or more genes and/or pathways, and/or introduce evolved and/or known mutations into depletor cells 912 to facilitate further strain differences between edited cells thereof.

The best performing cells 1102 exhibiting resistance to the novel antibiotic compounds produced by the co-encoded antibiotic pathways 951 are selected and verified at operation 1004. In certain embodiments, resistance is verified by, e.g., incubating the edited depletor cells 912 in the presence (via induction) or absence of the one or more novel antibiotic compounds above the minimum inhibitory concentration (MIC) of the cells with unedited resistance gene(s). The best performing cells 1102 may be further verified by analyzing growth rates thereof with and without antibiotic production expression, as well as by whole genome sequencing, mass spectrometry protein analysis, and/or metabolite analysis.

Thereafter, the best performing cells 1102 are cyclically edited and selected at operation 1006 to transfer the resistance edits into the genomes of the cells 1102 and increase resistance, which may also introduce additional targeted and genome-wide mutagenesis across one or more additional genes and/or pathways through single and/or combinatorial edits with designed mutagenesis libraries 1120. As a result, resistant antibiotic producer cells 1104 are formed, each having edited antibiotic production pathways 1151 facilitating production of novel antibiotic compound(s), one or more edited modification genes 1152, as well as one or more edited resistance pathways 953 to provide resistance to the novel antibiotic compounds produced. The resistant antibiotic producer cells 1104 may then be utilized during another round of barcoded modification of antibiotic production genes to produce further diversified and novel antibiotic compounds, as described below.

At operation 1008 shown in FIGS. 10 and 11B, the resistant antibiotic producer cells 1104 are subjected to editing with designed mutagenesis libraries 1122 to create novel library variant cells 1106 having further diversified antibiotic production pathways which produce diverse, novel antibiotic compounds upon induction. Accordingly, the designed mutagenesis libraries 1122 are designed for targeted mutations to the antibiotic production pathways 1151 previously edited during the method 800, as well as targeted mutations to the modification genes 1152 and nucleic acid barcodes to enable quantitative tracking of the edits. Similar to prior editing operations, the precise and targeted nature of the editing at operation 1008 enables multiplexed and genome-wide mutagenesis across one or more genes and/or pathways through single edits and/or combinatorial edits, thus facilitating maximum diversification of the antibiotic production pathways 1151. In certain embodiments, the designed mutagenesis libraries 1122 are designed for active site mutagenesis of antibiotic production enzymes encoded by the antibiotic production pathways 1151, active site mutagenesis of modification enzymes encoded by the modification genes 1152, as well as saturation mutagenesis.

The novel library variant cells 1106 are thereafter split into replicates of induced samples 1108 and uninduced samples 1110 at operation 1010, and are selected for reproducible cell depletion. Cell depletion indicates that a novel antibiotic compound different from the novel antibiotic compound produced in method 800 is being produced and is escaping the resistance afforded by the edited resistance pathways 1153. Depletor cells 1112, or cells being reproducibly depleted upon induction, are then utilized for further evaluation and antibiotic compound identification and isolation, and the method 1000 may be repeated cyclically to generate additional sets of novel antibiotic compounds and/or resistance pathways. Cyclical repetition of the method 1000 enables further modification to inserted genes and/or the original production pathways of the base strain to achieve maximum diversity of biological feasible antibiotic compound generation.

As during the method 800, sequencing of the bacterial cells, as well as the designed libraries for insertion thereto, is carried out before and/or after each of the operations of method 1000 to facilitate tracking of edits to the initial bacterial population. Thus, nucleic acid barcodes that are under-represented in the final cell population (e.g., after final sequencing) indicate novel antibiotic production pathways yielding novel antibiotic compounds that overcome the resistance pathways 1153.

In certain embodiments, sequence data collected from the methods 800, 1000 may have machine learning algorithms applied thereto to simulate further modifications to the base bacterial strain 900 during additional cycles of the methods 800 and/or 1000, as well as novel antibiotic compounds that may be produced thereby. Additionally, while mutations within evolution are typically derived from gene transfer or through natural evolution which is often limited to just one or two mutations per generation, the engineered approach here is unlinked from evolutionary constraints, whereby the mutations introduced in engineering would take evolution decades, centuries, or millennia to achieve the same result. Accordingly, the data collected from a several rounds of aforementioned methods may be utilized to predict and simulate evolutionary modifications to the base strain that would normally occur over the course of hundreds, thousands, or millions of years.

In summary, the present disclosure provides devices, instruments, and methods, including automated methods, for enhanced production and efficient screening of biosynthesized antibiotics. More particularly, the present disclosure provides for accelerated biosynthesis and screening on a single-cell scale.

Conventional methods of adaptive laboratory evolution (ALE) for biosynthesis of novel antibiotics require onerous, long-time-frame experiments with bacterial strains having elevated mutagenic rates. Utilizing the embodiments described herein, targeted and precise genome-wide edits can be delivered to transform and/or modify antibiotic production pathways and/or antibiotic resistance pathways of a single cell via exhaustive mutagenesis libraries, thus enabling exploration of evolutionary paths too distinct to be interrogated by other methods. Accordingly, the present disclosure enables simulation of the evolutionary antibiotics arms race that naturally occurs between microbes, and can further accelerate the process with machine learning and forward engineering to compress hundreds of millions of years of evolution into days, months, or years.

Although embodiments and examples herein are applied to antibacterial small molecule production and resistance pathways, the devices, instruments, and methods described may further be applied other engineered pathways, including but not limited to antifungal, anti-phage, and antiviral pathways.

While this invention is satisfied by embodiments in many different forms, as described in detail in connection with preferred embodiments of the invention, it is understood that the present disclosure is to be considered as exemplary of the principles of the invention and is not intended to limit the invention to the specific embodiments illustrated and described herein. Numerous variations may be made by persons skilled in the art without departure from the spirit of the invention. The scope of the invention will be measured by the appended claims and their equivalents. The abstract and the title are not to be construed as limiting the scope of the present invention, as their purpose is to enable the appropriate authorities, as well as the general public, to quickly determine the general nature of the invention. In the claims that follow, unless the term “means” is used, none of the features or elements recited therein should be construed as means-plus-function limitations pursuant to 35 U.S.C. § 112, ¶6.

1. A cell edited by automated editing, the cell comprising:
an antibiotic production pathway for producing an antibiotic compound; and
an antibiotic resistance pathway conferring resistance to the produced antibiotic compound.

2. The cell of claim 1, wherein the antibiotic production pathway is a heterologous pathway introduced into to the cell.

3. The cell of claim 1, where the antibiotic resistance pathway is a heterologous pathway introduced into to the cell.

4. The cell of claim 1, wherein one or more genes in the antibiotic production pathway are under the control of an inducible promoter.

5. The cell of claim 1, wherein the cell is a microbial cell.

6. The cell of claim 5, wherein the cell is a bacterial cell or fungal cell.

7. A method of synthesizing an antibacterial compound, comprising:
introducing at least one of an antibiotic production pathway and an antibiotic resistance pathway into each cell of an initial population such that each cell has both the antibiotic production pathway and the antibiotic resistance pathway, the antibiotic production pathway controlled by an inducible promoter;
identifying one or more cells of the initial population in which the antibiotic resistance pathway confers resistance to an initial antibiotic compound produced by the antibiotic production pathway when induced;
editing the antibiotic production pathway of each identified cell exhibiting resistance to the initial antibiotic compound;
selecting one or more cells having the edited antibiotic production pathway that deplete upon induction of the edited antibiotic production pathway, the depletion of the selected cells indicating production of a novel antibiotic compound escaping resistance conferred by the antibiotic resistance pathway; and
isolating the novel antibiotic compound from the selected cells.

8. The method of claim 7, further comprising:
upon selecting one or more cells that deplete upon induction of the edited antibiotic production pathway, editing the resistance pathway of the selected cells; and
identifying one or more selected cells in which the edited antibiotic resistance pathway confers resistance to the novel antibiotic compound produced by the edited antibiotic production pathway when induced.

9. The method of claim 8, further comprising:
upon identifying one or more selected cells in which the edited antibiotic resistance pathway confers resistance to the novel antibiotic compound, editing the previously-edited antibiotic production pathway of the selected cells to form a further edited antibiotic production pathway; and
screening the selected cells having the further edited antibiotic production pathway for cells that deplete upon induction of the further edited antibiotic production pathway, the depletion of the selected cells indicating production of a second novel antibiotic compound escaping resistance conferred by the edited antibiotic resistance pathway.

10. The method of claim 7, wherein introducing at least one of the antibiotic production pathway and the antibiotic resistance pathway is carried out via nucleic-acid guided editing utilizing designed mutagenesis libraries having nucleic acid barcodes.

11. The method of claim 10, wherein the cells of the initial population are sequenced before and after introduction of the at least one of the antibiotic production pathway and the antibiotic resistance pathway, and wherein editing events are tracked via the presence or absence of the nucleic acid barcodes.

12. The method of claim 10, wherein the designed mutagenesis libraries are further designed to introduce targeted and genome-wide mutagenesis across one or more genes other than the antibiotic production pathway and the antibiotic resistance pathway through edits at up to hundreds of thousands of loci of the cells of the initial population.

13. The method of claim 7, wherein editing the antibiotic production pathway of each identified cell exhibiting resistance is carried out via nucleic-acid guided editing utilizing designed mutagenesis libraries having nucleic acid barcodes.

14. The method of claim 13, wherein each of the identified cells is sequenced before and after editing the antibiotic production pathway, and wherein editing events are tracked via the presence or absence of the nucleic acid barcodes.

15. The method of claim 13, wherein the designed mutagenesis libraries are further designed to introduce targeted and genome-wide mutagenesis across one or more genes other than the antibiotic production pathway and the antibiotic resistance pathway through edits at up to hundreds of thousands of loci of the identified cells.

16. The method of claim 7, wherein the antibiotic production pathway is a heterologous pathway introduced into to the cell.

17. The method of claim 7, where the antibiotic resistance pathway is a heterologous pathway introduced into to the cell.

18. The method of claim 7, wherein the initial population comprises microbial cells.

19. The method of claim 14, wherein the initial population comprises bacterial cells.

20. The method of claim 7, wherein each cell is sequenced before and/or after each edit applied thereto, and wherein data collected from sequencing of the cells is applied to one or more machine learning algorithms to simulate future edits to the cells.
